(19) United States
(12) Patent Application Publication
(10) Pub. No.: US 2006/0257439 A1
(43) Pub. Date: Nov. 16, 2006

(54) Cleansing compositions with color changing indicator

(76) Inventors: Ram W. Sabnis, Eagan, MN (US); Timothy D. Kehoe, St. Paul, MN (US); Robert James Balchunis, St. Paul, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/391,004

(22) Filed: Mar. 28, 2006

Related U.S. Application Data
(60) Provisional application No. 60/734,218, filed on Nov. 7, 2005.
(60) Provisional application No. 60/752,673, filed on Dec. 21, 2005.
(60) Provisional application No. 60/666,028, filed on Mar. 29, 2005.
(60) Provisional application No. 60/696,872, filed on Jul. 6, 2005.
(60) Provisional application No. 60/711,183, filed on Aug. 25, 2005.

(51) Int. Cl. A61K 8/35

(52) U.S. Cl. 424/401

(57) ABSTRACT

The invention describes compositions that can indicate whether hands have been washed for an approximate predetermined period of time. The composition includes an acid-base indicator and a delivery system.

### CROSS REFERENCE TO RELATED APPLICATIONS

This application claims benefit under 35 U.S.C. § 119(e) to U.S. Ser. Nos. 60/696,872, filed Jul. 6, 2005 (Attorney docket number 186573/US), entitled “Color Changing Compositions and Articles” and 60/711,183, filed Aug. 25, 2005 (Attorney docket number 186978/US), entitled “Substituted Phenol-Based Aqueous Indicators”.

The application also claims benefit under 35 U.S.C. § 119(e) to U.S. Ser. Nos. 60/734,218, filed Nov. 7, 2005 (Attorney docket number 187226/US), entitled “Color Changing Hand Soap Composition” and 60/752,673, filed Dec. 21, 2005 (Attorney docket number 187226/US/2), entitled “Color Changing Hand Soap Composition”, the contents of which are incorporated herein by reference in their entirety.

### FIELD OF THE INVENTION

The invention relates generally hand soaps that when applied at first have a color that then later disappears as the hands are rubbed together over a time that is appropriate to effectuate proper cleansing.

### BACKGROUND OF THE INVENTION

Soap and water are effective cleaners and, depending on ingredients, can be effective in fighting bacteria and other causes of illness. In many cases, effective cleaning and disease control occur only after certain periods of time spent washing. Children, and many adults, do not always take the appropriate time needed to effectively clean their hands.

It is, therefore, quite important for children and adults to spend adequate time cleaning hands and learn the correct way of completing a key hygiene task.

In order for proper hand cleaning habits to form, the teaching and monitoring must be done in a non-threatening and natural manner. One way of accomplishing this would be to introduce an element of fun and novelty so that children and adults enjoy completing the task while building better hygiene habits. Another way would be give them a sense of accomplishment by providing a feedback signal they can easily understand and associate with correctly completing the task.

There remains a need for a soap product that will give an indication of when sufficient use has occurred. It is an object of this invention to provide such a use indicating soap. Conventional soaps used for hand washing do not indicate whether the soap has been used for an appropriate amount of time for the process to be effective. As a result, hands are often washed for too short an amount of time for the process to be effective in cleansing hands. Properly washing your hands is one of the best ways to prevent infection and the spread of diseases. Doctors, nurses and other people who work in medical settings have to wash their hands frequently to avoid spreading infective agents between patients or to themselves.

Those who prepare food must keep clean hands so they do not put germs into the food they are making. Also, childcare workers must wash their hands often so they do not make children sick. In addition to professionals whose hygiene is regulated, individuals should be conscious of the germs on their hands. Turning doorknobs, handling money and coughing may transfer bacteria and viruses to one's hands, which can then be spread to other people's hands or to your mouth and eyes and cause infection. Anyone who prepares food for others can infect others if his or her hands are not properly cleaned. Many of the diseases spread this way are more inconvenient than a simple cold. Some examples are the flu, hepatitis A and typhoid. See Wisconsin Department of Health and Family Services, “Hand Washing,” available at <http://healthlink.mcw.edu/article/955074416.html> Aug. 29, 2002.

Approaches ensuring effective hand cleansing include antibacterial hand rubs. One drawback with such hand rubs is that such rubs may dry hands more than washing with soap. Another approach known involves applying a dye that glows under a black light to hands prior to washing hands, washing hands, then analyzing the effectiveness of the hand-washing process by examining the hands under a black light. In addition, some hospitals use electronic devices that alert an employee if the employee's hands were not effectively washed. Such approaches may be expensive and fail to achieve widespread use.

Therefore, a need exists for an effective way to cleanse a person's hands while providing an indication that the cleansing is effective.

### BRIEF SUMMARY OF THE INVENTION

In response to the discussed difficulties and problems encountered prior to the present invention, a new cleaning aid has been developed wherein the aid contains an indicator that provides a color change detectible by a user after a period of time of rubbing hands together. The observable color change may occur in from a finite time to at most 5 minutes or more particularly about 45 seconds, or still more particularly between 25 and 35 seconds.

If the novel cleaning aid is a soap, the soap is preferably made from a first component including liquid soap and a second component of a pH sensitive dye. The components are mixed together to produce a novel color changing soap. The soap will remain colored in a container or dispenser and turn colorless or to another color after it is applied to a users hands and a period of time has passed rubbing their hands together. The embodiments allow a determination of whether hands have been washed for at least an approximate predetermined period of time. The color change can signal that the appropriate period of time has lapsed. The predetermined period of time can be varied depending on the hand washing needs. The soap may change color from colored to colorless, color to color or colorless to color. The soap is a self-contained single-phase system needing no other chemicals or acids to trigger the color change. Suitable acid-base indicators are those described throughout the specification vide infra.

In other embodiments, the acid-base indicator chemistry can be utilized in nail polish, shaving cream/gel

The present invention can be used in a number of settings including, but not limited to, private homes, hospitals, childcare centers, nursing homes, schools, restaurants, airports, and food-preparation and food-processing establishments.

The details of one or more embodiments of the invention are set forth in the description below. Other features, objects, and advantages of the invention will be apparent from the description and from the claims.

### DETAILED DESCRIPTION

In general, a composition for indicating whether a portion of a body has been washed for an approximate predetermined period of time includes pH sensitive dye that changes color as the pH of the environment surrounding the indicator changes. The colored composition can be delivered in the form of a body wash, such as a bar, a liquid hand soap, shower gel or a shampoo. Such modes of delivery, bars, liquid soaps, shower gels and shampoos are known in the art and can be utilized with the pH sensitive dyes described throughout the specification.

As used herein, “bodywash” encompasses any type of cleansing vehicle applied to the body. Exemplary forms of cleansing vehicles include, but are not limited to, liquid, bar, gel, foam, aerosol or pump spray, cream, lotion, stick, powder, or incorporated into a patch or a towelette. In addition, soapless cleansers may be used as well. The bodywash can be made into any suitable product form. Thus, as used herein, “bodywash” includes, but is not limited to, a soap including liquid and bar soap; a shampoo; a hair conditioner; a shower gel; including an exfoliating shower gel; a foaming bath product (e.g. gel, soap or lotion); a milk bath; a soapless cleanser, including a gel cleanser, a liquid cleanser and a cleansing bar; moist towelletes; a body lotion; a body spray, mist or gel; bath effervescent tablets (e.g., bubble bath); a hand and nail cream; a bath/shower gel; a shower cream; a depilatory cream; a shaving product e.g. a shaving cream, gel, foam or soap, an after-shave, after-shave moisturizer; and combinations thereof, and any other composition used for cleansing or post-cleansing application to the body, including the skin and hair. Especially useful as bodywashes in the invention are soaps, e.g., liquid soaps and bar soaps, and shampoos.

For example, the color changing compositions herein can be formulated can be formulated, for example, as bar soaps such those as disclosed in U.S. Pat. Nos. 3,993,722 and 3,070,547, liquid personal cleaning compositions such as those in U.S. Pat. Nos. 4,387,040; 4,673,523; 3,697,644; 3,932,610; 4,031,306; 4,061,602; 4,387,040; 4,917,823; 5,296,158; 4,338,211; 4,190,549 and 4,861,507 and as shampoos such as disclosed in U.S. Pat. Nos. 4,345,080, 4,704,272 and 4,741,855, the contents of which are incorporated herein by reference in their entirety for all purposes.

Bar soaps are prepared from water-soluble soaps including sodium, potassium, ammonium and alkanol-ammonium (e.g., mono-, di-, triethanolammonium) salts of higher fatty acids (e.g. C₁₀-C₂₄) as a major component. Particularly useful are the fatty acids derived from coconut oil and tallow, i.e., sodium and potassium tallow, and coconut soaps. The soap can be prepared through conventional milling and optional plodding steps well known in the art. The soap begins typically as a kettle soap which is dried and then mixed with desired adjuvants as perfume, fillers, color indicator (acid-base indicator) emollients, water, salt, etc., and is thereafter milled into chips, ribbons, pellets, or noodles that can be formed into bars. Preferred major soap constituents are tallow and coconut soaps at weight ratios of tallow to coconut soap ranging from 95:5 to 5:95. In some embodiments, soaps comprise from about 40% to 90% by weight tallow soap and/or those which comprise about 10% to 60% coconut soaps.

Liquid personal cleaning compositions are well known in the art such as U.S. Pat. Nos. 4,387,040; 4,673,523; 3,697,644; 3,932,610; 4,031,306; 4,061,602; 4,387,040; 4,917,823; 5,296,158; 4,338,211; 4,190,549 and 4,861,507, the contents of which are incorporated herein in their entirety for all purposes.

Traditional liquid soaps based upon unsaturated fatty acids (coco, oleic, soya, lauric, myristic etc.). The soaps usually consist of a blend of coco soap and oleates (or soaps derived from soya or other vegetable oils rich in oleic acid). It has found that stable concentrated liquid aqueous soap compositions can be obtained if the soap comprises a mixture of potassium salts of lauric acid and myristic acid and coconut diethanolamide with a viscosity controlling component that can include a mixture of coconut diethanolamide and sodium sulfate.

Typically a liquid hand soap of the invention comprises from about 1 to about 40 weight % surfactant. In particular, from about 2 to 20 weight %, most particularly from about 3 to 10 weight %, surfactant is employed, selected from the group of anionic, nonionic, zwitterionic, ampholytic and cationic surfactants. The liquid hand soap can further comprise emollient (up to about 30 weight %) and minor amounts of perfume, indicator dye, solvent, and opacifier.

In general, a shampoo includes from about 5 to 60 weight % surfactant, generally selected from lauryl sulfate, isoethionate, acyl amidobetaine, alkyl glyceryl ether sulfonate, and alkyl ether sulfate, coamidopropyl betaine, coamide DEA, polyethylene glycol polymers, including disterates therof, and indicator dye. Optional ingredients are suds booster, conditioner, perfume and/or anti-dandruff agent.

Shower gels can be formulated with, for example, sodium C₁₄-C₁₆ olefin sulfonate solutions, ammonium lauryl sulfate, cocamide DEA, salt, citric acid and the indicator dye.

The pH sensitive dyes used in the present invention are generally colored under basic condition and change color or fade to clear in non-basic condition. Acid pH sensitive dyes which are colored on alkaline pH side (pH >7) and turn clear on acidic pH (pH <7) are most useful. Typically, the pH sensitive dyes are colored at pH between about 9 and 10, and turn clear at pH between about 6 and 8.

Representative examples of pH sensitive dyes useful in the compositions of the present invention include, but are not limited to, picric acid, matius yellow, 2,6-dinitrophenol, 2,4-dinitrophenol, phenacetolin, 2,5-dinitrophenol, isopicramic acid, o-nitrophenol, m-nitrophenol, p-nitrophenol, 6,8-dinitro-2,4-(1H,3H)quinazolinedione, nitroamine, ethyl bis(2,4-dinitrophenyl)-acetate, 2,4,6-trinitrotoluene, 1,3,5-trinitrobenzene, 2,4,6-tribromobenzoic acid, 2-(p-dimethylaminophenyl)azopyridine, metanil yellow, p-methyl red, 4-phenylazodiphenylamine, benzopurpurin 4B, tropaeolin OO, fast garnet GBC base, alizarin yellow R, benzyl orange, m-methyl red, 4-(m-tolyl)-azo-N,N-dimethyl-aniline, oil yellow II, methyl orange, ethyl orange, hessian purple N, congo red, N-pnehyl-1-naphthyl-aminoazobenzene-p-sulfonic acid, 4-(4′-dimethylamino-1′-naphthyl)-azo-3-methoxy-benzenesulfonic acid, p-ethoxychrysoidine, α-naphthyl red, chrysoidine, 1-naphthylaminoazobenzene-p-sulfonic acid, methyl red, 2-(p-dimethylaminophenyl)-azopyridine, ethyl red, propyl red, N-phenyl-1-naphthyl-aminoazo-o-carboxybenzene, nitrazol yellow, brilliant yellow, brilliant yellow S, orange II, propyl-o-naphthyl orange, orange I, orange IV, hessian, Bordeaux, diazo violet, α-naphthol violet, alizarin yellow GG, chrome orange GR, sulfone acid blue R, lanacyl violet BF, tropaeolin O, orange G, crystal violet, methyl violet B, malachite green, brilliant green, ethyl violet, methyl violet 6B, ethyl/methyl green, basic fuchsine, acid, fuchsine, patent blue V, alkali blue, aniline blue, o-naphthol benzein, pentamethoxy red, hexamethoxy red, tetrabromophenolphthalein ethyl ester K salt, tetraiodophenolsulfophthlein, bromochlorophenol blue, bromocresol green, chlorocresol green, chlorophenol red, bromocresol purple, sulfonaphthyl red, bromophenol red, dibromophenol-tetrabromophenol-sulfophthlein, bromothymol blue, aurin, phenol red, o-cresol benzein, o-cresol red, α-naphtholphthlein, m-cresol purple, p-xylenol blue, thymol blue, phenoltetrachlorophthlein, o-cresolphthalein, α-naphtholbenzein, phenoltetraiodophthlein, phenolphthalein, thymolphthlein, eosin Y, erythrosine B, erythrosine, galleon, brilliant cresyl blue, resazurin, lacmoid, litmus, azolitmus, azolitmin, neutral red, nile blue 2B, nile blue A, hematoxylin, quinaldine red, pinachrome, indo-oxine, quinoline blue, bis-5-bromovanillidenecyclohexanone, bis-(2′-hydroxystyryl)ketone, curcumin, bis-(4-hydroxy-3-ethoxy-benzylidene)-cyclohexanone, thiazole yellow G, alizarin blue B, alizarin red S, carminic acid, alizarin orange, alizarin, rufianic acid, rufianic blue, alizarin blue SWR, and indigocarmine and salts thereof. With the suitable selection of pH sensitive dyes, it is possible to produce any color. Additionally, combinations of two or more indicators may be used.

The pH sensitive dyes are preferably in the form of a salt, such as a sodium salt generated by reacting the indicator with an alkali or alkaline metal hydroxide, such as sodium hydroxide, or other such metallic base (for example a metallic alkoxide such as sodium ethoxide) so as to permit the resultant salt to be solubilized in an aqueous system and produce a color. Many substances that readily absorb carbon dioxide and moisture from the air and are soluble in water, alcohol, and glycerin may work in the system.

pH sensitive dyes are usually effective when present in small amounts in the compositions of the invention but generally are present in amounts from about 0.01% up to about 20% by weight, from about 0.5% to about 10% by weight and from about 0.8% to about 8% by weight of the total weight of the composition.

It should be understood that the term “comprising” (or comprises) includes the more restrictive terms consisting of and consisting essentially of.

The metal (i.e., sodium) salt form of the pH sensitive dyes are basic and remain basic in surfactant solutions such as Sodium Lauryl Sulfate. The amount of a metal hydroxide, such as sodium hydroxide and acid-base indicator can be used to control the fading time of the color after application.

Particular phthaleins useful in the invention have the formula (I):

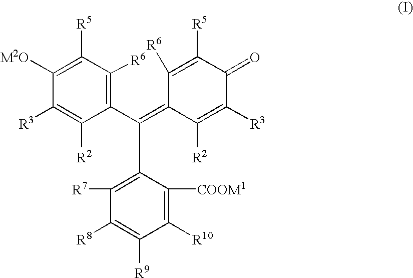

wherein R², R³, R⁵, R⁶, R⁷, R⁸, R⁹ and R¹⁰ are each, independently of one another, selected from the group consisting of hydrogen, —OH, —SH, —CN, —NO₂, halo, fluoro, chloro, bromo, iodo, lower alkyl, substituted lower alkyl, lower heteroalkyl, substituted lower heteroalkyl, cycloalkyl, substituted cycloalkyl, cycloheteroalkyl, substituted cycloheteroalkyl, lower haloalkyl, monohalomethyl, dihalomethyl, trihalomethyl, trifluoromethyl, lower alkylthio, substituted lower alkylthio, lower alkoxy, substituted lower alkoxy, methoxy, substituted methoxy, lower heteroalkoxy, substituted lower heteroalkoxy, cycloalkoxy, substituted cycloalkoxy, cycloheteroalkoxy, substituted cycloheteroalkoxy, lower haloalkoxy, monohalomethoxy, dihalomethoxy, trihalomethoxy, trifluoromethoxy, amino, lower di- or monoalkylamino, substituted lower di- or monoalkylamino, aryl, substituted aryl, aryloxy, substituted aryloxy, phenoxy, substituted phenoxy, arylalkyl, substituted arylalkyl, arylalkyloxy, substituted arylalkyloxy, benzyl, benzyloxy, heteroaryl, substituted heteroaryl, heteroaryloxy, substituted heteroaryloxy, heteroarylalkyl, substituted heteroarylalkyl, heteroarylalkyloxy, substituted heteroarylalkyloxy, carboxyl, lower alkoxycarbonyl, substituted lower alkoxycarbonyl, aryloxycarbonyl, substituted aryloxycarbonyl, arylalkyloxycarbonyl, substituted arylalkyloxycarbonyl, carbamate, substituted carbamate, carbamoyl, substituted carbamoyl, sulfamoyl or substituted sulfamoyl.

Alternatively, R² and R³, R⁵ and R⁶ or R² and R³, and R⁵ and R⁶ can form cyclic ring structures that are heterocyclic, heteroaromatic, aromatic or nonaromatic and can contain one or more heteroatoms to form, for example, a quinoline, napthalene, etc.

Additionally, R⁷ and R⁸, R⁸ and R⁹, R⁹ and R¹⁰ or combinations thereof can form cyclic ring structures that are heterocyclic, heteroaromatic, aromatic or nonaromatic and can contain one or more heteroatoms to form, for example, a quinoline, napthalene, etc.

Optionally, one of the carbons connected to R², R³, R⁵ or R⁶ can be substituted with a nitrogen atom.

M¹ and M² are each independently a hydrogen atom, a metal ion or an ammonium ion.

In certain embodiments, R² is selected from the group consisting of hydrogen, nitro, amino and alkyl; R³ is selected from the group consisting of hydrogen, phenyl, alkyl, nitro, acetamido and alkoxy; R⁵ is selected from the group consisting of hydrogen, halo, and alkyl; and R⁶ is selected from the group consisting of hydrogen and alkyl.

In other certain embodiments, R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

In other embodiments, R², R³, R⁴, R⁵, R⁶, R⁷, R⁸, R⁹ and R¹⁰ are all hydrogen atoms, or R² is hydrogen, R³ is Me, and R⁵, R⁶, R⁷, R⁸, R⁹ and R¹⁰ are all hydrogen atoms, or R² is Me, R³ is a hydrogen atom, R⁵ is an iso-propyl group and R⁶, R⁷, R⁸, R⁹ and R¹⁰ are all hydrogen atoms, or R² is H, R³ is Me, R⁵ is Br and R⁶, R⁷, R⁸, R⁹ and R¹⁰ are all hydrogen atoms, or R² is Me, R³ is Br, R⁵ is an isopropyl and R⁶, R⁷, R⁸, R⁹ and R¹⁰ are all hydrogen atoms. In certain embodiments, one or more of these compounds may be excluded from certain aspects of the invention.

In still other embodiments, R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R³ is H, R⁵ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms, or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰, are all hydrogen atoms and R³ is sec-butyl, or R², R³, R⁵, R⁶, R⁷, R⁸, R⁹, R¹⁰ are all hydrogen atoms and R³ is nitro.

In particular, at least one of M¹ or M² is a metal or an ammonium ion.

It should be understood, that the salt form of the indicator can be isolated prior to use or prepared in situ. Ideally, the salt is formed as a mono-salt or a di-salt, meaning that excess base is not present and either 1 or 2 equivalents of base react with the acidic protons of the indicator.

The following table provides phthaleins of particular interest.

| R² | R³ | R⁵ | R⁶ | Color |
|---|---|---|---|---|
| H | phenyl | H | H | purple |
| H | i-propyl | i-propyl | H | violet |
| H | Me | H | Me | blue |
| H | OMe | OMe | H | teal |
| H | Me | Me | H | purple |
| H | Et | H | H | magenta |
| H | i-propyl | H | H | pink |
| H | OMe | H | H | blue |
| Me | Me | Me | H | teal |
| H | Me | H | H | magenta |
| H | i-propyl | H | Me | blue |
| H | Me | Br | H | purple |
| H | i-propyl | Br | Me | teal |
| H | sec-butyl | H | H | pink |
| H | NO₂ | H | H | yellow |

In another aspect, the acid-base indicator can be a substituted phenol of formula (II):

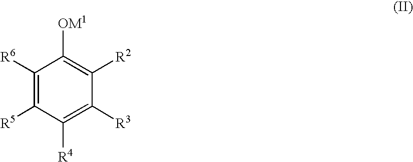

wherein R², R³, R⁵, R⁶ and M¹ are as defined above and R⁴ is selected from the same group as R², R³, R⁵ and R⁶.

Alternatively, R² and R³, R³ and R⁴, R⁴ and R⁵, or R⁵ and R⁶ can form cyclic ring structures that are heterocyclic, heteroaromatic, aromatic or nonaromatic and can contain one or more heteroatoms to form, for example, a quinoline, napthalene, etc.

In one aspect, one or more of R² through R⁶, independently, is a nitro (—NO₂) group and the remaining R groups are selected from those provided above.

Additionally, substituted hydrazides are useful in the compositions of the invention and can have one of two formulae:

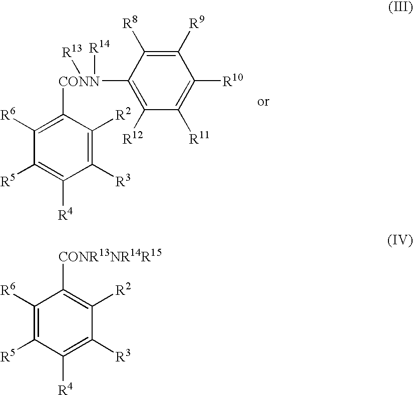

wherein R² through R⁶ are as defined above and R⁸ through R¹² are the same substituents as R² through R⁶. R¹³, R¹⁴ and R¹⁵ (if present) are each, independently of one another, a hydrogen atom, an alkyl group, a substituted alkyl group, any aryl group or a substituted aryl group.

In certain embodiments for compound formulae (II), R¹³ and R¹⁴ are hydrogen atoms and for compound formulae (III), R¹³, R¹⁴ and R¹⁵ are all hydrogen atoms.

In certain aspects, compounds of formulae (III) can have one or more hydroxyl groups, which can be deprotonated to form a salt. For example, formulae (IIIa) provides one isomer where a hydroxyl is present at the R² position as a salt. M² is as defined above for M¹. It should be understood that one or more of R² through R¹² could have a hydroxyl at that given position, and that hydroxyl could be in a salt form.

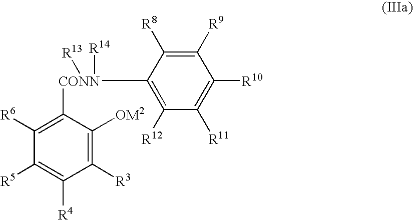

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 15 carbon atoms (C₁-C₁₅ alkyl), more preferably from 1 to 10 carbon atoms (C₁-C₁₀ alkyl) and even more preferably from 1 to 6 carbon atoms (C₁-C₆ alkyl or lower alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” by itself or as part of another substituent refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group comprises from 1 to 6 carbon atoms (C1-C6 alkyldiyl). Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno,” by itself or as part of another substituent, refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3) alkyleno. Also preferred are straight-chain saturated alkano groups, e.g., methano, ethano, propano, butano, and the like.

“Alkoxy,” by itself or as part of another substituent, refers to a radical of the formula —OR, where R is an alkyl or cycloalkyl group as defined herein. Representative examples alkoxy groups include, but are not limited to, methoxy, ethoxy, propoxy, isopropoxy, butoxy, tert-butoxy, cyclopropyloxy, cyclopentyloxy, cyclohexyloxy and the like.

“Alkoxycarbonyl,” by itself or as part of another substituent, refers to a radical of the formula —C(O)-alkoxy, where alkoxy is as defined herein.

“Alkylthio,” by itself or as part of another substituent, refers to a radical of the formula —SR, where R is an alkyl or cycloalkyl group as defined herein. Representative examples of Alkylthio groups include, but are not limited to, methylthio, ethylthio, propylthio, isopropylthio, butylthio tert-butylthio, cyclopropylthio, cyclopentylthio, cyclohexylthio, and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 6 to 20 carbon atoms (C₆-C₂₀ aryl), more preferably from 6 to 15 carbon atoms (C₆-C₁₅ aryl) and even more preferably from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl, more preferably, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl, and even more preferably, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Aryloxy,” by itself or as part of another substituent, refers to a radical of the formula —O-aryl, where aryl is as defined herein.

“Arylalkyloxy, by itself or as part of another substituent, refers to a radical of the formula —O-arylalkyl, where arylalkyl is as defined herein.

“Aryloxycarbonyl,” by itself or as part of another substituent, refers to a radical of the formula —C(O)—O-aryl, where aryl is as defined herein.

“Carbamoyl,” by itself or as part of another substituent, refers to a radical of the formula —C(O)NR′R″, where R′ and R″ are each, independently of one another, selected from the group consisting of hydrogen, alkyl and cycloalkyl as defined herein, or alternatively, R′ and R″, taken together with the nitrogen atom to which they are bonded, form a 5-, 6- or 7-membered cycloheteroalkyl ring as defined herein, which may optionally include from 1 to 4 of the same or different additional heteroatoms selected from the group consisting of O, S and N.

“Compounds of the invention” refers to compounds encompassed by the various descriptions and structural formulae disclosed herein. The compounds of the invention may be identified by either their chemical structure and/or chemical name. When the chemical structure and chemical name conflict, the chemical structure is determinative of the identity of the compound. The compounds of the invention may contain one or more chiral centers and/or double bonds and therefore may exist as stereoisomers, such as double-bond isomers (i.e., geometric isomers), rotamers, enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds of the invention may also exist in several tautomeric forms including the enol form, the keto form and mixtures thereof. Accordingly, the chemical structures depicted herein encompass all possible tautomeric forms of the illustrated compounds. The compounds of the invention may also include isotopically labeled compounds where one or more atoms have an atomic mass different from the atomic mass conventionally found in nature. Examples of isotopes that may be incorporated into the compounds of the invention include, but are not limited to, ²H, ³H, ¹¹C, ¹³C, ¹⁴C, ¹⁵N, ¹⁸O, ¹⁷O, ³¹P, ³²P, ³⁵S, ¹⁸F and ³⁶Cl. Compounds of the invention may exist in unsolvated forms as well as solvated forms, including hydrated forms and as N-oxides. In general, the hydrated, solvated and N-oxide forms are within the scope of the present invention. Certain compounds of the present invention may exist in multiple crystalline or amorphous forms. In general, all physical forms are equivalent for the uses contemplated by the present invention and are intended to be within the scope of the present invention.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. Preferably, the cycloalkyl group comprises from 3 to 10 ring atoms (C₃-C₁₀ cycloalkyl) and more preferably from 3 to 7 ring atoms (C₃-C₇ cycloalkyl).

“Cycloheteroalkyl,” by itself or as part of another substituent, refers to a saturated or unsaturated cyclic alkyl radical in which one or more carbon atoms (and optionally any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidone, quinuclidine, and the like. Preferably, the cycloheteroalkyl group comprises from 3 to 10 ring atoms (3-10 membered cycloheteroalkyl) and more preferably from 5 to 7 ring atoms (5-7 membered cycloheteroalkyl).

A cycloheteroalkyl group may be substituted at a heteroatom, for example, a nitrogen atom, with a lower alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of “cycloheteroalkyl.” A cycloheteralkyl group may be attached to the remainder of the molecule via a ring carbon atom or a ring heteroatom.

“Dialkylamino” or “Monoalkylamino,” by themselves or as part of other substituents, refer to radicals of the formula —NRR and —NHR, respectively, where each R is independently selected from the group consisting of alkyl and cycloalkyl, as defined herein. Representative examples of dialkylamino groups include, but are not limited to, dimethylamino, methylethylamino, di-(1-methylethyl)amino, (cyclohexyl)(methyl)amino, (cyclohexyl)(ethyl)amino, (cyclohexyl)(propyl)amino and the like. Representative examples of monalkylamino groups include, but are not limited to, methylamino, ethylamino, propylamino, isopropylamino, cyclohexylamino, and the like.

“Halogen” or “Halo,” by themselves or as part of another substituent, refer to a fluoro, chloro, bromo and/or iodo radical.

“Haloalkyl,” by itself or as part of another substituent, refers to an alkyl group as defined herein in which one or more of the hydrogen atoms is replaced with a halo group. The term “haloalkyl” is specifically meant to include monohaloalkyls, dihaloalkyls, trihaloalkyls, etc. up to perhaloalkyls. The halo groups substituting a haloalkyl can be the same, or they can be different. For example, the expression “(C₁-C₂) haloalkyl” includes 1-fluoromethyl, 1-fluoro-2-chloroethyl, difluoromethyl, trifluoromethyl, 1-fluoroethyl, 1,1-difluoroethyl, 1,2-difluoroethyl, 1,1,1-trifluoroethyl, perfluoroethyl, etc. “Haloalkyloxy,” by itself or as part of another substituent, refers to a group of the formula —O-haloalkyl, where haloalkyl is as defined herein.

“Heteroalkyl,” “Heteroalkanyl,” “Heteroalkenyl,” “Heteroalkynyl,” “Heteroalkyldiyl” and “Heteroalkyleno,” by themselves or as part of other substituents, refer to alkyl, alkanyl, alkenyl, alkynyl, alkyldiyl and alkyleno groups, respectively, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, O, S, N, Si, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Examples of such heteroalkyl, heteroalkanyl, heteroalkenyl and/or heteroalkynyl groups include —CH₂—CH₂—O—CH₃, —CH₂—CH₂—NH—CH₃, —CH₂—CH₂—N(CH₃)—CH₃, —CH₂—S—CH₂, —CH₃, —CH₂—CH₂—S(O)—CH₃, —CH₂—CH₂—S(O)₂—CH₃, —CH═CH—O—CH₃, —CH₂—CH═N—O—CH₃, and —CH₂—CH₂—O—C═CH. For heteroalkyldiyl and heteroalkyleno groups, the heteratom or heteratomic group can also occupy either or both chain termini. For such groups, no orientation of the group is implied.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group comprises from 5 to 20 ring atoms (5-20 membered heteroaryl), more preferably from 5 to 10 ring atoms (5-10 membered heteroaryl). Preferred heteroaryl groups are those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Metal ion” or “Metal Salt” refers to a salt of a compound of the invention which is made with counterions understood in the art to be generally acceptable for pharmaceutical uses and which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like (see, e.g., Berge et al., 1977, J. Pharm. Sci. 66:1-19).

“Pharmaceutically acceptable vehicle” refers to a diluent, adjuvant, excipient or carrier with which a compound of the invention is administered.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O—, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b} —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR)NR^{c}R^{c}, —OC(O)R^{b}, —OC(S)R^{b}, —OC(O)O—, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Sulfamoyl,” by itself or as part of another substituent, refers to a radical of the formula —S(O)₂NR′R″, where R′ and R″ are each, independently of one another, selected from the group consisting of hydrogen, alkyl and cycloalkyl as defined herein, or alternatively, R′ and R″, taken together with the nitrogen atom to which they are bonded, form a 5-, 6- or 7-membered cycloheteroalkyl ring as defined herein, which may optionally include from 1 to 4 of the same or different additional heteroatoms selected from the group consisting of O, S and N.

Methods of Synthesis

The particular phthaleins described above can be obtained via synthetic methods illustrated below. It should be understood that in R², R³, R⁵, R⁶, R⁷, R⁸, R⁹ and R¹⁰, are as previously defined for structural formulae (I) and (Ia).

Starting materials useful for preparing compounds of the invention and intermediates thereof are commercially available or can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995). Other methods for synthesis of the compounds described herein and/or starting materials are either described in the art or will be readily apparent to the skilled artisan.

A typical synthesis is depicted in Scheme I, wherein 2 equivalents of a phenol or phenol equivalent are condensed with 1 equivalent of a phthalic anhydride or equivalent under essentially acid anhydrous conditions.

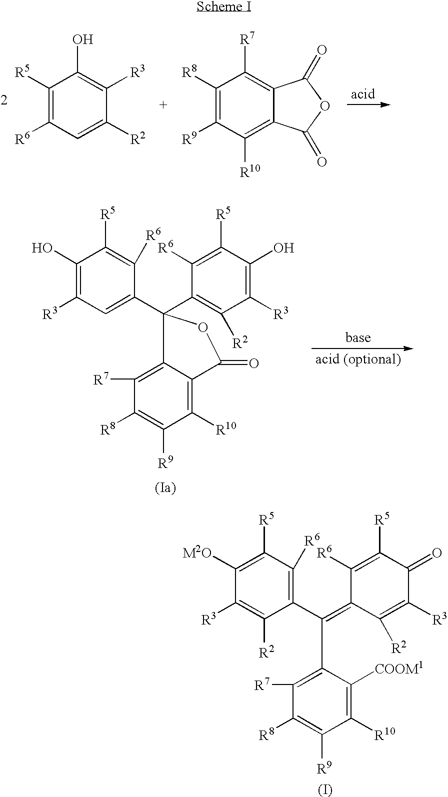

Generally, the phenol and anhydride are condensed in the presence of an acid under anhydrous conditions. For example, polyphosphoric acid and zinc chloride can be utilized. The carbon atom at 4-position-position with respect to the aromatic hydroxyl group must not be substituted as it is necessary for reaction. Polyphosphoric acid acts as a condensing agent as well as reaction medium. The reaction with only polyphosphoric acid afforded tarry products but when very small amount of zinc chloride was added to polyphosphoric acid, clean product was isolated. Very small amount of zinc chloride was found to increase yield and purity of the product. Polyphosphoric acid can be replaced with orthophosphoric acid, chlorosulfonic acid, methane sulfonic acid, trifluoroacetic acid or other acids under anhydrous conditions. Suitable solvents include non-protic solvents known in the art such as tetrahydrofuran, dioxane, methylene chloride, ether, etc.

The reaction proceeds with the formation of an isobenzofuranone (Ia), which is then treated with a base under aqueous conditions. The salt can be isolated or the solution can be acidified to produce the protonated phenol/carboxylic acid. For example, one molar equivalent of Ia was condensed with either two molar equivalent of sodium hydroxide in 85% ethanol or two molar equivalent of sodium ethoxide in ethanol. The products are generally solids and can be easily purified via filtration, crystallization, and other methods known in the art.

Suitable phenols include, but are not limited to 2-nitrophenol, 3-nitrophenol, 2-chlorophenol, 3-chlorophenol, 2-bromophenol, 3-bromophenol, 2-iodophenol, 3-iodophenol, 2-fluorophenol, 3-fluorophenol, 2-aminophenol, 3-aminophenol, 2-acetamidophenol, 3-acetamidophenol, 2-cyanophenol, 3-cyanophenol, 2-methylphenol, 3-methylphenol, 2-ethylphenol, 3-ethylphenol, 2-proylphenol, 3-proylphenol, 2-isoproylphenol, 3-isoproylphenol, 2-butylphenol, 3-butylphenol, 2-isobutylphenol, 3-isobutylphenol, 2-pentylphenol, 3-pentylphenol 2-hexylphenol, 3-hexylphenol, 2-heptylphenol, 3-heptylphenol, 2-octylphenol, 3-octylphenol, 2-nonylphenol, 3-nonylphenol, 2-decylphenol, 3-decylphenol, 2-decylphenol, 2-methoxyphenol, 3-methoxyphenol, 2-ethoxyphenol, 3-ethoxyphenol, 2-propoxyphenol, 3-propoxyphenol, 2-isopropoxyphenol, 3-isopropoxyphenol, 2-butoxyphenol, 3-butoxyphenol, 2-isobutoxyphenol, 3-isobutoxylphenol, 2-allylphenol, 3-allylphenol, 2-vinylphenol, 3-vinylphenol, 2-phenylphenol, 3-phenylphenol, 2-phenoxyphenol, 3-phenoxyphenol, 2-cyclopropylphenol, 3-cyclopropylphenol, 2-cyclobutylphenol, 3-cyclobutylphenol, 2-cyclopentylphenol, 3-cyclopentylphenol, 2-cyclohexylphenol, 3-cyclohexylphenol, 2-cycloheptylphenol, 3-cycloheptylphenol, 2-cyclooctylphenol, 3-cyclooctylphenol, 2-cyclononylphenol, 3-cyclononylphenol, 2-cyclodecylphenol, 3-cyclodecylphenol, 2,3-dinitrophenol, 2,5-dinitrophenol, 2,6-dinitrophenol, 2,3-dimethylphenol, 2,5-dimethylphenol, 2,6-dimethylphenol, 2,3-diethylphenol, 2,5-diethylphenol, 2,6-diethylphenol, 2,3-diproplylphenol, 2,5-dipropylphenol, 2,6-dipropylphenol, 2,3-diisoproplylphenol, 2,5-diisopropylphenol, 2,6-diisopropylphenol, 2,3-dibutylphenol, 2,5-dibutylphenol, 2,6-dibutylphenol, 2,3-diisobutylphenol, 2,5-diisobutylphenol, 2,6-diisobutylphenol, 2,3-dipentylphenol, 2,5-dipentylphenol, 2,6-dipentylphenol, 2,3-dihexylphenol, 2,5-dihexylphenol, 2,6-dihexylphenol, 2,3-diheptylphenol, 2,5-diheptylphenol, 2,6-diheptylphenol, 2,3-dioctylphenol, 2,5-dioctylphenol, 2,6-dioctylphenol, 2,3-dinonylphenol, 2,5-dinonylphenol, 2,6-dinonylphenol, 2,3-didecylphenol, 2,5-didecylphenol, 2,6-didecylphenol, 2,3-dimethoxyphenol, 2,5-dimethoxyphenol, 2,6-dimethoxyphenol, 2,3-diethoxyphenol, 2,5-diethoxyphenol, 2,6-diethoxyphenol, 2,3-dipropoxyphenol, 2,5-dipropoxyphenol, 2,6-dipropoxyphenol, 2,3-diisopropoxyphenol, 2,5-diisopropoxyphenol, 2,6-diisopropoxyphenol, 2,3-dibutoxyphenol, 2,5-dibutoxyphenol, 2,6-dibutoxyphenol, 2,3-diisobutoxyphenol, 2,5-diisobutoxyphenol, 2,6-diisobutoxyphenol, 2,3-dipentoxyphenol, 2,5-dipentoxyphenol, 2,6-dipentoxyphenol, 2,3-dihexoxyphenol, 2,5-dihexoxyphenol, 2,6-dihexoxyphenol, 2,3-diheptoxyphenol, 2,5-diheptoxyphenol, 2,6-diheptoxyphenol, 2,3-dioctoxyphenol, 2,5-dioctoxyphenol, 2,6-dioctoxyphenol, 2,3-dinonoxyphenol, 2,5-dinonoxyphenol, 2,6-dinonoxyphenol, 2,3-didecyloxyphenol, 2,5-didecyloxyphenol, 2,6-didecyloxyphenol, 2,3-dichlorophenol, 2,5-dichlorophenol, 2,6-dichlorophenol, 2,3-dibromophenol, 2,5-dibromophenol, 2,6-dibromophenol, 2,3-diiodophenol, 2,5-diiodophenol, 2,6-diiodophenol, 2,3-difluorophenol, 2,5-difluorophenol, 2,6-difluorophenol, 2,3-diaminophenol, 2,5-diaminophenol, 2,6-diaminophenol, 2,3-diacetamidophenol, 2,5-diacetamidophenol, 2,6-diacetamidophenol, 2,3-dicyanophenol, 2,5-dicyanophenol, 2,6-dicyanophenol, 2,3-diallylphenol, 2,5-diallylphenol, 2,6-diallylphenol, 2,3-divinylphenol, 2,5-divinylphenol, 2,6-divinylphenol, 2,3-diphenylphenol, 2,5-diphenylphenol, 2,6-diphenylphenol, 2,3-diphenoxyphenol, 2,5-diphenoxyphenol, 2,6-diphenoxyphenol, 2,3-dicycloproylphenol, 2,5-dicyclopropylphenol, 2,6-dicyclopropylphenol, 2,3-dicyclobutylphenol, 2,5-dicyclobutylphenol, 2,6-dicyclobutylphenol, 2,3-dicyclopentylphenol, 2,5-dicyclopentylphenol, 2,6-dicyclopentylphenol, 2,3-dicyclohexylphenol, 2,5-dicyclohexylphenol, 2,6-dicyclohexylphenol, 2,3-dicycloheptylphenol, 2,5-dicycloheptylphenol, 2,6-dicycloheptylphenol, 2,3-dicyclooctylphenol, 2,5-dicyclooctylphenol, 2,6-dicyclooctylphenol, 2,3-dicyclononylphenol, 2,5-dicyclononylphenol, 2,6-dicyclononylphenol, 2,3-dicyclodecylphenol, 2,5-dicyclodecylphenol, 2,6-dicyclodecylphenol, 2,3,5-trimethylphenol, 2,3,6-trimethylphenol 2,3,5-triethylphenol, 2,3,6-triethylphenol, 2,3,5-tripropylphenol, 2,3,6-tripropylphenol, 2,3,5-tributylphenol, 2,3,6-tributylphenol, 2,3,5-trichlorophenol, 2,3,6-trichlorophenol, 2,3,5-tribromophenol, 2,3,6-tribromophenol, 2,3,5-triiodophenol, 2,3,6-triiodophenol, 2,3,5-trifluorophenol, 2,3,6-trifluorophenol, 2,3,5-trivinylphenol, 2,3,6-trivinylphenol, 2,3,5-triallylphenol, 2,3,6-triallylphenol, 2,3,5-triphenylphenol, 2,3,6-triphenylphenol, 2,3,5-triphenoxyphenol, 2,3,6-triphenoxyphenol, 2,3,5-trimethoxyphenol, 2,3,6-trimethoxyphenol, 2,3,5-triethoxyphenol, 2,3,6-triethoxyphenol, 2,3,5-tripropoxyphenol, 2,3,6-tripropoxyphenol, 2,3,5-tributoxyphenol, 2,3,6-tributoxyphenol, 2,3,5-trinitrophenol, 2,3,6-trinitrophenol, 2,3,5-triaminophenol, 2,3,6-triaminophenol, 2,3,5-triacetamidophenol, 2,3,6-triacetamidophenol, 2,3,5-tricyanophenol, 2,3,6-tricyanophenol, 3-(N,N-diethylamino)phenol, 2-tert-butyl-5-methylphenol, 2-tert-butyl-6-methylphenol, 3-methyl-2-nitrophenol, 5-methyl-2-nitrophenol, 6-methyl-2-nitrophenol, 3-ethyl-2-nitrophenol, 5-ethyl-2-nitrophenol, 6-ethyl-2-nitrophenol, 3-methoxyl-2-nitrophenol, 5-methoxy-2-nitrophenol, 6-methoxy-2-nitrophenol, 1-naphthaol, 2-naphthaol, 2-nitro-1-naphthol, 3-nitro-1-naphthol, 5-nitro-1-naphthol, 6-nitro-1-naphthol, 7-nitro-1-naphthol, 8-nitro-1-naphthol, 2-methyl-1-naphthol, 3-methyl-1-naphthol, 5-methyl-1-naphthol, 6-methyl-1-naphthol, 7-methyl-1-naphthol, 8-methyl-1-naphthol, 2-methoxy-1-naphthol, 3-methoxy-1-naphthol, 5-methoxy-1-naphthol, 6-methoxy-1-naphthol, 7-methoxy-1-naphthol, 8-methoxy-1-naphthol, 2-chloro-1-naphthol, 3-chloro-1-naphthol, 5-chloro-1-naphthol, 6-chloro-1-naphthol, 7-chloro-1-naphthol, 8-chloro-1-naphthol, 2-bromo-1-naphthol, 3-bromo-1-naphthol, 5-bromo-1-naphthol, 6-bromo-1-naphthol, 7-bromo-1-naphthol, 8-bromo-1-naphthol, 2-iodo-1-naphthol, 3-iodo-1-naphthol, 5-iodo-1-naphthol, 6-iodo-1-naphthol, 7-iodo-1-naphthol, 8-iodo-1-naphthol, 2-fluoro-1-naphthol, 3-fluoro-1-naphthol, 5-fluoro-1-naphthol, 6-fluoro-1-naphthol, 7-bromo-1-naphthol, 8-fluoro-1-naphthol, 2-cyano-1-naphthol, 3-cyano-1-naphthol, 5-cyano-1-naphthol, 6-cyano-1-naphthol, 7-cyano-1-naphthol, 8-cyano-1-naphthol, 8-hydroxyquinaldine and 2-quinoxalinol.

The term “phenol equivalent” is intended to include those compounds where, as described above, R² and R³, for example, form an aromatic, heterocyclic, or non-aromatic ring. Suitable compounds include naphthols for example.

Suitable phthalic anhydrides include but are not limited to phthalic anhydride, 3-nitrophthalic anhydride, 4-nitrophthalic anhydride, 5-nitrophthalic anhydride, 6-nitrophthalic anhydride, 3-chlorophthalic anhydride, 4-chlorophthalic anhydride, 5-chlorophthalic anhydride, 6-chlorophthalic anhydride, 3-bromophthalic anhydride, 4-bromophthalic anhydride, 5-bromophthalic anhydride, 6-bromophthalic anhydride, 3-iodophthalic anhydride, 4-iodophthalic anhydride, 5-iodophthalic anhydride, 6-iodophthalic anhydride, 3-fluorophthalic anhydride, 4-fluorophthalic anhydride, 5-fluorophthalic anhydride, 6-fluorophthalic anhydride, 3-methylphthalic anhydride, 4-methylphthalic anhydride, 5-methylphthalic anhydride, 6-methylphthalic anhydride, 3-ethylphthalic anhydride, 4-ethylphthalic anhydride, 5-ethylphthalic anhydride, 6-ethylphthalic anhydride, 3-methoxyphthalic anhydride, 4-methoxyphthalic anhydride, 5-methoxyphthalic anhydride, 6-methoxyphthalic anhydride, 3-cyanophthalic anhydride, 4-cyanophthalic anhydride, 5-cyanophthalic anhydride, 6-cyanophthalic anhydride, 3-aminophthalic anhydride, 4-aminophthalic anhydride, 5-aminophthalic anhydride, 6-aminophthalic anhydride, 3-acetamidophthalic anhydride, 4-acetamidophthalic anhydride, 5-acetamidophthalic anhydride, 6-acetamidophthalic anhydride, 3,4,5,6-tetrachlorophthalic anhydride, 3,4,5,6-tetrabromophthalic anhydride, 3,4,5,6-tetraiodophthalic anhydride, 3,4,5,6-tetrafluorophthalic anhydride, 3,4,5,6-tetranitrophthalic anhydride, 3,4,5,6-tetramethylphthalic anhydride, 3,4,5,6-tetraethylphthalic anhydride, 3,4,5,6-tetramethoxyphthalic anhydride, 3,4,5,6-tetracyanophthalic anhydride, 3,4,5,6-tetraminophthalic anhydride, 3,4,5,6-tetraacetamidophthalic anhydride, naphthalic anhydride, 2-chloronaphthalic anhydride, 3-chloronaphthalic anhydride, 4-chloronaphthalic anhydride, 5-chloronaphthalic anhydride, 6-chloronaphthalic anhydride, 7-chloronaphthalic anhydride, 2-bromonaphthalic anhydride, 3-bromonaphthalic anhydride, 4-bromonaphthalic anhydride, 5-bromonaphthalic anhydride, 6-bromonaphthalic anhydride, 7-bromonaphthalic anhydride, 2-iodonaphthalic anhydride, 3-iodonaphthalic anhydride, 4-iodonaphthalic anhydride, 5-iodonaphthalic anhydride, 6-iodonaphthalic anhydride, 7-iodonaphthalic anhydride, 2-fluoronaphthalic anhydride, 3-fluoronaphthalic anhydride, 4-fluoronaphthalic anhydride, 5-fluoronaphthalic anhydride, 6-fluoronaphthalic anhydride, 7-fluoronaphthalic anhydride, 2-nitronaphthalic anhydride, 3-nitronaphthalic anhydride, 4-nitronaphthalic anhydride, 5-nitronaphthalic anhydride, 6-nitronaphthalic anhydride and 7-nitronaphthalic anhydride.

The term “phthalic anhydride equivalent” is intended to include those compounds where, as described above, R⁷ and R⁸, for example, form an aromatic, heterocyclic, or non-aromatic ring. Suitable compounds include naphthols for example.

Synthesis of Phenols and Hydrazides

The compounds of the invention may be obtained via synthetic methods illustrated below. It should be understood that R², R³, R⁴, R⁵, R⁶, R⁷, R⁸, R⁹, R¹⁰, R¹¹, R¹², R¹³, R¹⁴ and R¹⁵ are as previously defined for structural formulae (II), (III), (IIIa) and (IV).

Starting materials useful for preparing compounds of the invention and intermediates thereof are commercially available or can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995). Other methods for synthesis of the compounds described herein and/or starting materials are either described in the art or will be readily apparent to the skilled artisan.

A typical synthesis for substituted phenols is depicted in Scheme II, wherein a phenol is treated with a base to form the phenolic salt. Advantageously, the phenolic salts are water soluble, which is useful in the applications detailed throughout the specification.

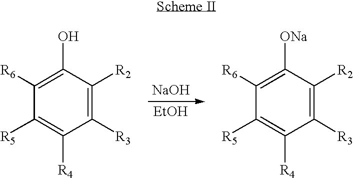

Generally, the phenol mixed with the base and the salt is formed. The solution may be heated to facilitate the rate of reaction.

Suitable phenols include, but are not limited to 2-nitrophenol, 3-nitrophenol, 4-nitrophenol, 2-chlorophenol, 3-chlorophenol, 4-chlorophenol, 2-bromophenol, 3-bromophenol, 4-bromophenol, 2-iodophenol, 3-iodophenol, 4-iodophenol, 2-aminophenol, 3-aminophenol, 4-aminophenol, 2-cyanophenol, 3-cyanophenol, 4-cyanophenol, 2-vinylphenol, 3-vinylphenol, 4-vinylphenol, 2,3-dichlorophenol, 2,4-dichlorophenol, 2,5-dichlorophenol, 2,6-dichlorophenol, 2,3-dibromophenol, 2,4-dibromophenol, 2,5-dibromophenol, 2,6-dibromophenol, 2,3-diiodophenol, 2,4-diiodophenol, 2,5-diiodophenol, 2,6-diiodophenol, 2,3-diaminophenol, 2,4-diaminophenol, 2,5-diaminophenol, 2,6-diaminophenol, 2,3-dicyanophenol, 2,4-dicyanophenol, 2,5-dicyanophenol, 2,6-dicyanophenol, 2,3-divinylphenol, 2,4-divinylphenol, 2,5-divinylphenol, 2,6-divinylphenol, 2,3-diphenylphenol, 2,3,4-trichlorophenol, 2,3,5-trichlorophenol, 2,3,6-trichlorophenol, 2,3,4-tribromophenol, 2,3,5-tribromophenol, 2,3,6-tribromophenol, 2,3,4-triiodophenol, 2,3,5-triiodophenol, 2,3,6-triiodophenol, 2,3,4-trivinylphenol, 2,3,5-trivinylphenol, 2,3,6-trivinylphenol, 2,3,4-trinitrophenol, 2,3,5-trinitrophenol, 2,3,6-trinitrophenol, 2,3,4-triaminophenol, 2,3,5-triaminophenol, 2,3,6-triaminophenol, 2,3,4-tricyanophenol, 2,3,5-tricyanophenol, 2,3,6-tricyanophenol, 3-(N,N-diethylamino)phenol, 3-methyl-2-nitrophenol, 5-methyl-2-nitrophenol, 6-methyl-2-nitrophenol, 3-ethyl-2-nitrophenol, 5-ethyl-2-nitrophenol, 6-ethyl-2-nitrophenol, 3-methoxyl-2-nitrophenol, 5-methoxy-2-nitrophenol, 6-methoxy-2-nitrophenol, 2-nitro-1-naphthol, 3-nitro-1-naphthol, 4-nitro-1-naphthol, 5-nitro-1-naphthol, 6-nitro-1-naphthol, 7-nitro-1-naphthol, 8-nitro-1-naphthol, 2-chloro-1-naphthol, 3-chloro-1-naphthol, 4-chloro-1-naphthol, 5-chloro-1-naphthol, 6-chloro-1-naphthol, 7-chloro-1-naphthol, 8-chloro-1-naphthol, 2-bromo-1-naphthol, 3-bromo-1-naphthol, 4-bromo-1-naphthol, 5-bromo-1-naphthol, 6-bromo-1-naphthol, 7-bromo-1-naphthol, 8-bromo-1-naphthol, 2-iodo-1-naphthol, 3-iodo-1-naphthol, 4-iodo-1-naphthol, 5-iodo-1-naphthol, 6-iodo-1-naphthol, 7-iodo-1-naphthol, 8-iodo-1-naphthol, 2-cyano-1-naphthol, 3-cyano-1-naphthol, 4-cyano-1-naphthol, 5-cyano-1-naphthol, 6-cyano-1-naphthol, 7-cyano-1-naphthol, 8-cyano-1-naphthol and 8-hydroxyquinaldine.

The term “phenol equivalent” is intended to include those compounds where, as described above, R² and R³, for example, form an aromatic, heterocyclic, or non-aromatic ring. Suitable compounds include naphthols for example.

A typical synthesis of hydrazines is depicted in Scheme III, where a hydrazine (NH₂NH—R¹⁵, wherein R¹⁵ can be a hydrogen atom or as described above) and an ester are condensed to form the hydrazide.

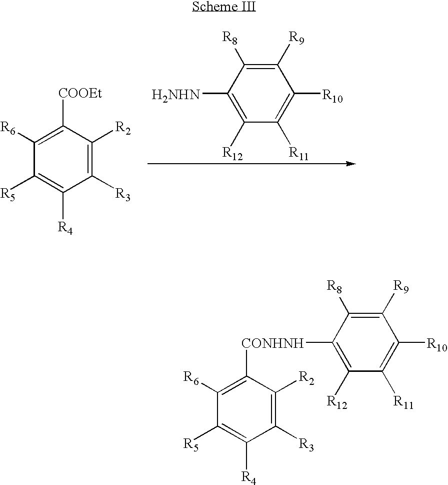

Typically the ester and the hydrazine are combined in a solvent, such as a protic solvent, e.g., an alcohol, such as ethanol, and heated, e.g., to reflux. Upon cooling, the hydrazide generally precipitates from solution and can be collected.

Suitable salicylic derivatives include, but not limited to salicylic acid, 3-methylsalicylic acid, 4-methylsalicylic acid, 5-methylsalicylic acid, 6-methylsalicylic acid, 3-ethylsalicylic acid, 4-ethylsalicylic acid, 5-ethylsalicylic acid, 6-ethylsalicylic acid, 3-propylsalicylic acid, 4-propylsalicylic acid, 5-propylsalicylic acid, 6-propylsalicylic acid, 3-isopropylsalicylic acid, 4-isopropylsalicylic acid, 5-isopropylsalicylic acid, 6-isopropylsalicylic acid, 3-butylsalicylic acid, 4-butylsalicylic acid, 5-butylsalicylic acid, 6-butylsalicylic acid, 3-isobutylsalicylic acid, 4-isobutylsalicylic acid, 5-isobutylsalicylic acid, 6-isobutylsalicylic acid, 3-methoxysalicylic acid, 4-methoxysalicylic acid, 5-methoxysalicylic acid, 6-methoxysalicylic acid, 3-ethoxysalicylic acid, 4-ethoxysalicylic acid, 5-ethoxysalicylic acid, 6-ethoxysalicylic acid, 3-propoxysalicylic acid, 4-propoxysalicylic acid, 5-propoxysalicylic acid, 6-propoxysalicylic acid, 3-butoxysalicylic acid, 4-butoxysalicylic acid, 5-butoxysalicylic acid, 6-butoxysalicylic acid, 3-nitrosalicylic acid, 4-nitrosalicylic acid, 5-nitrosalicylic acid, 6-nitrosalicylic acid, 3-chlorosalicylic acid, 4-chlorosalicylic acid, 5-chlorosalicylic acid, 6-chlorosalicylic acid, 3-bromosalicylic acid, 4-bromosalicylic acid, 5-bromosalicylic acid, 6-bromosalicylic acid, 3-iodosalicylic acid, 4-iodosalicylic acid, 5-iodosalicylic acid, 6-iodoosalicylic acid, 3-fluorosalicylic acid, 4-fluorosalicylic acid, 5-fluorosalicylic acid, 6-fluorosalicylic acid, 3-aminosalicylic acid, 4-aminosalicylic acid, 5-aminosalicylic acid, 6-aminosalicylic acid, 3-acetamidosalicylic acid, 4-acetamidosalicylic acid, 5-acetamidosalicylic acid, 6-acetamidosalicylic acid, 3-cyanosalicylic acid, 4-cyanosalicylic acid, 5-cyanosalicylic acid, 6-cyanosalicylic acid, 3-sulfosalicylic acid, 4-sulfosalicylic acid, 5-sulfosalicylic acid, 6-sulfosalicylic acid, 3,5-dimethylsalicylic acid, 3,5-diethylsalicylic acid, 3,5-dipropylsalicylic acid, 3,5-dibutylsalicylic acid, 3,5-dimethoxysalicylic acid, 3,5-diethoxysalicylic acid, 3,5-dipropoxysalicylic acid, 3,5-dibutoxysalicylic acid, 3,5-dichlorosalicylic acid, 3,5-dibromosalicylic acid, 3,5-diiodosalicylic acid, 3,5-difluorosalicylic acid, 3,5-dinitrosalicylic acid, 3,5-diaminosalicylic acid, 3,5-diacetamidosalicylic acid, 3,5-dicyanosalicylic acid, 3,5-disulfosalicylic acid, substituted/unsubstituted alkyl salicylic acid, substituted/unsubstituted alkoxy salicylic acid, substituted/unsubstituted aryl salicylic acid, substituted/unsubstituted cycloalkyl salicylic acid and substituted/unsubstituted hetaryl salicylic acid.

Suitable hydrazines include but not limited to hydrazine hydrate, 4-nitrophenylhydrazine, 3-nitrophenylhydrazine, 2-nitrophenylhydrazine, 4-nitrobenzoic hydrazide, 3-nitrobenzoic hydrazide, 2-nitrobenzoic hydrazide, p-toluenesulfonylhydrazide, m-toluenesulfonylhydrazide, o-toluenesulfonylhydrazide, 2,4-dinitrophenylhydrazine (2,4-DNP), 1-naphthoic hydrazide, 2-naphthoic hydrazide, nicotinic hydrazide, substituted/unsubstituted alkyl hydrazide, substituted/unsubstituted alkoxy hydrazide, substituted/unsubstituted aryl hydrazide, substituted/unsubstituted cycloalkyl hydrazide and substituted/unsubstituted hetaryl hydrazide.

Another important component to the compositions of the invention is the inclusion of a surfactant. Suitable surfactants include anionic, cationic, nonionic or zwitterionic compounds and combinations thereof. The surfactant can be either polymeric or non-polymeric.

The term “surfactant” is recognized in the relevant art to include those compounds which modify the nature of surfaces, e.g. reducing the surface tension of water. Surfactants are generally classified into four types: cationic (e.g. modified onium salts, where part of the molecule is hydrophilic and the other consists of straight or branches long hydrocarbon chains such as hexadecyltrimethyl bromide), anionic, also known as amphiphatic agents (e.g., alkyl or aryl or alkylarylsulfonates, carboxylates, phosphates), nonionic (e.g., polyethylene oxides, alcohols) and ampholytic or amphoteric (e.g. dodecyl-beta-alanine, such that the surfactant contains a zwitterionic group). One or more surfactants can be used in the present invention.

Cationic surfactants useful as surface tension reducing agents in the present invention include long chain hydrocarbons which contain quaternarized heteroatoms, such as nitrogen. Suitable cationic surfactants include quaternary ammonium compounds in which typically one of the groups linked to the nitrogen atom is a C12-C18 alkyl group and the other three groups are short chained alkyl groups.

Anionic surfactants (amphiphatic agents) are characterized by a single lipophilic chain and a polar head group which can include sulfate, sulfonate, phosphate, phosphonate and carboxylate. Exemplary compounds include linear sodium alkyl benzene sulfonate (LAS), linear alkyl sulfates and phosphates, such as sodium lauryl sulfate (SLS) and linear alkyl ethoxy sulfates. Additional examples of anionic surfactants include substituted ammonium (e.g., mono-, di-, and tri-ethanolammonium), alkali metal and alkaline earth metal salts of C6-C20 fatty acids and rosin acids, linear and branched alkyl benzene sulfonates, alkyl ether sulfates, alkane sulfonates, olefin sulfonates, hydroxyalkane sulfonates, fatty acid monoglyceride sulfates, alkyl glyceryl ether sulfates, acyl sarcosinates. acyl N-methyltaurides, and alkylaryl sulfonated surfactants, such as alkylbenezene sulfonates.

Nonionic surfactants do not dissociate but commonly derive their hydrophilic portion from polyhydroxy or polyalkyloxy structures. Suitable examples of polyhydroxy (polyhydric) compounds include ethylene glycol, butylene glycol, 1,3-butylene glycol, propylene glycol, glycerine, 2-methyl-1,3-propane diol, glycerol, mannitol, corn syrup, beta-cyclodextrin, and amylodextrin. Suitable examples of polyalkyloxy compounds include diethylene glycol, dipropylene glycol, polyethylene glycols, polypropylene glycols and glycol derivatives.

Other suitable nonionic surfactants include other linear ethoxylated alcohols with an average length of 6 to 16 carbon atoms and averaging about 2 to 20 moles of ethylene oxide per mole of alcohol; linear and branched, primary and secondary ethoxylated, propoxylated alcohols with an average length of about 6 to 16 carbon atoms and averaging 0-10 moles of ethylene oxide and about 1 to 10 moles of propylene oxide per mole of alcohol; linear and branched alkylphenoxy (polyethoxy) alcohols, otherwise known as ethoxylated alkylphenols, with an average chain length of 8 to 16 carbon atoms and averaging 1.5 to 30 moles of ethylene oxide per mole of alcohol; and mixtures thereof.

Additionally, suitable nonionic surfactants include polyoxyethylene carboxylic acid esters, fatty acid glycerol esters, fatty acid and ethoxylated fatty acid alkanolamides. Block copolymers of propylene oxide and ethylene oxide, and block polymers of propylene oxide and ethylene oxide with propoxylated ethylene diamine are also included as acceptable nonionic surfactants. Semi-polar nonionic surfactants like amine oxides, phosphine oxides, sulfoxides, and their ethoxylated derivatives are included within the scope of the invention.

Suitable amphoteric and zwitterionic surfactants which contain an anionic water-solubilizing group, a cationic group and a hydrophobic organic group include amino carboxylic acids and their salts, amino dicarboxylic acids and their salts, alkylbetaines, alkyl aminopropylbetaines, sulfobetaines, alkyl imidazolinium derivatives, certain quaternary ammonium compounds, certain quaternary phosphonium compounds and certain tertiary sulfonium compounds

Examples of anionic, nonionic, cationic and amphoteric surfactants that are suitable for use in the present invention are described in Kirk-Othmer, Encyclopedia of Chemical Technology, Third Edition, Volume 22, pages 347-387, and McCutcheon's Detergents and Emulsifiers, North American Edition, 1983, both of which are incorporated herein by reference.

Typical concentration ranges of surfactant that are useful in the present compositions are from about 0.01 parts by weight to about 90 parts by weight, from about 0.5 part by weight to about 50 parts by weight, and from about 1 parts by weight to about 10 parts by weight.

In one aspect, surfactants useful in the compositions of the invention include, but are not limited to, cellulose ethers or mixtures with other surfactants, which are water soluble. Cellulose ether surfactants have unique foaming properties which make them ideal for foaming hand soap applications. Cellulose ethers used in the present invention include methyl cellulose, ethyl cellulose, propyl cellulose, butyl cellulose, higher alkyl, aryl, alkoxy, cycloalkyl celluloses, hydroxypropyl cellulose, hydroxybutyl cellulose or mixtures thereof.

Commercial cellulose ether surfactants include, but are not limited to, Methocel A4M, methyl cellulose, Methocel F4M, hydroxypropyl methylcellulose, Methocel K4M, hydroxypropyl methylcellulose, manufactured by Dow Chemical Co., Mildland, Mich.; Natrosol, hydroxyethyl cellulose, Klucel, hydroxypropyl cellulose, Aqualon Cellulose Gum, sodium carboxymethyl cellulose, Hercules Inc., Wilmington, Del.; Elfacos CD 481, ethyl 2-hydroxyethyl ether cellulose, manufactured by Akzo Nobel, Chicago, Ill.

Cellulose ether surfactants are generally present in amounts from about 1% up to about 40% by weight in the compositions of the invention. Suitable concentrations of cellulose ether surfactants are in the range of about 2% to about 30% by weight and from about 3% to about 8% by weight. A particularly useful cellulosic ether surfactant in the compositions is Methocel A4M.

In another aspect, alkanolamide or a mixture with other surfactants can be used in the compositions of the invention. Alkanolamides are commercially available and are the reaction products of one or more fatty acids having 12 or more carbon atoms and a lower alkanolamime. Typical alkanolamides are formed by reaction between stearic, mystiric, lauric acid or mixtures thereof with mono-, di-, and/or iso-propanolamine.

Alkanolamides can be present in the compositions of the invention in the ranges generally described throughout the application but generally are present in amounts from about 0% up to about 10% by weight. Suitable ranges include from about 1% to about 6% by weight and in particular from about 1.5% to about 4% by weight.

In one embodiment, the alkanolamide surfactants of the present invention include, but are not limited to, Ninol 55LL, diethanolamine, Ninol 40CO, cocamide DEA, Ninol 30LL, lauramide DEA, manufactured by Stepan Co., Northfield, Ill.; Colamid C, cocamide DEA, Colamid 0071-J, alkanolamide, manufactured by Colonial Chemical Inc., S. Pittsburgh, Tenn. In one aspect, the alkanolamides are Ninol 55LL, and Colamid C.

Exemplary sulfosuccinates that can be employed in the present compositions include, but are not limited to, Stepan-Mild SL3-BA, disodium laureth sulfosuccinate, Stepan-Mild LSB, sodium lauryl sulfosuccinate, manufactured by Stepan Co., Northfield, Ill., Lankropol 4161L, sodium fatty alkanolamide sulfosuccinate and Colamate-DSLS, disodium laureth sulfosuccinate, manufactured by Colonial Chemical Inc., S. Pittsburgh, Tenn.

Suitable betaines that can be employed in the present compositions include, but are not limited to, Miracare BC-27, cocamidopropyl betaine and Miranol Ultra C-37, sodium cocoampho acetate, manufactured by J & S Chemical Co., Weston, Fla.

Suitable sulfates that can be employed in the present compositions include Rhodapex ES-2, sodium laureth sulfate, J & S Chemical Co., Weston, Fla.; Witcolate WAQ, sodium alkyl sulfate, manufactured by Akzo Nobel, Chicago, I and Colonial-SLS, sodium lauryl sulfate, manufactured by Colonial Chemical Inc., S. Pittsburgh, Tenn. Colonial-SLS surfactant is a combination of lauryl sulfate, C10-C16 alkyl alcohols, sodium salts and C10-C16 alcohols.

A suitable nonionic surfactant that can be employed in the present compositions is Triton H-66, alkyl aryl alkoxy potassium salt, manufactured by Dow Chemical Co., Mildland, Mich.

In one particular embodiment, the surfactant used is a combination of an ether based surfactant, such as a cellulose ether surfactant and an sodium alkyl sulfate, such as sodium lauryl sulfate.

In a particular embodiment, the surfactant is a combination of Methocel A4M (4 weight percent in aqueous solution) and sodium lauryl sulfate (30 weight percent in aqueous solution) in a (1:1 ratio) with a concentration range of from about 1 part by weight to about 10 parts by weight of the total weight of the composition. In particular aspects, the total weight of the ether surfactant and the alkyl sulfate surfactant of the total weight of the composition is between about 3 percent and about 8 percent by weight, more particularly between about 3 percent and about 5 percent by weight, and in particular about 5 percent by weight.

In another embodiment, the surfactant used is a combination of an alkanolamide and a mixture of an alkyl betaine and/or an alkyl sulfonate.

In a particular embodiment, the surfactant is a combination of Colamid C and Miracare BC27 which is a mixture of Surfactant blend include sodium trideceyl sulfate, water, PEG 80 sorbitant laurate, cocamidopropyl betaine, sodium lauroamphoacetate, PEG 150 distearate, sodium laureth-13 carboxylate, glycerin, citric acid, tetrasodium EDTA, quaternium-15. Generally, the combination of the alkanolamide and alkylsulfonate/betaine is in the range of between about 1:1 to about 1:7, more particularly between about 1:1 to about 2:7 and more particularly about 2:7. Generally, the combination of the two surfactants comprises a concentration between about 3 and about 10 percent by weight of the total weight of the composition, and more particularly between about 5 and about 10 percent by weight of the total weight of the composition, and in particular about 9 percent of the total weight of the composition.

In another embodiment, the invention pertains to a waterless organic solvent based hand sanitizer that includes one or more of the acid-base indicators described throughout the application. Suitable solvents include those listed throughout the application, including for example, isopropyl alcohol, glycols and other low molecular weight alcohols known in the art.

Waterless hand cleaner formulations include an organic solvent that is compatible with skin, and a quantity of water. A surfactant mixed with the organic solvent and the quantity of water forms a gelatinous emulsion therebetween. The invention also provides a method for cleaning and sanitizing a skin surface. The method includes the application to a skin surface of a waterless hand cleaner containing one of the acid-base indicators described herein. After allowing sufficient contact time between the hand cleaner and the skin surface, a cleaned skin surface results. Typically, thirty seconds to three minutes is sufficient to clean and sanitize the skin surface. Thereafter, the excess hand cleaner is removed from the now cleaned and sanitized skin surface by wiping or water rinsing.

As used herein, a “waterless hand cleaner” is defined to include a composition that removes soil and/or grease from a skin surface absent the addition of water during the hand cleansing process even though water is optionally used thereafter as a rinsing agent.

Waterless formulations are known in the art and generally include lipophilic solvents such as mineral spirits, C₁-C₃₀ oils, C₁-C₃₀ alcohols, C₁-C₃₀ fatty acids, and terpenoids; emollients such as lanolin, surfactants and detergents; fragrances; perfumes; thixotropic agents, water; chelating agents such as EDTA; bases such as caustic soda; antioxidants such as tocopherol acetate, ascorbate and BHT; thickeners such as propylene glycol; film forming plant extracts such as aloe vera; cellulosic material; starch; preservatives; and inorganic fillers and antimicrobials such as ZnO.

For example, a waterless hand cleaner formulation contains an emulsifiable organic solvent that is compatible with human skin contact. The emulsifiable organic solvent is present from about 2 to about 70 total weight percent and is capable of solubilizing lipophilic greases and soils. An organic solvent operative herein includes straight chain or branched chain aliphatic hydrocarbons having from about 6 to about 24 carbon atoms, alkaline glycol, alkaline glycol ether, dibasic ester, and alkyl-substituted aromatics. Specific examples of operative organic solvents illustratively include kerosene, naphtha, petroleum distillate, toluene, d-limonene, phenoxy ethanol, octanol, methyl soyate, cetyl acetate, and acetylated lanolin alcohol.

A surfactant, such as those described herein, is also present in a waterless hand cleaner formulation to form an emulsion between the emulsifiable organic solvent and water also present in the formulation. The surfactant can be a water soluble or water dispersible nonionic, anionic, cationic, or an amphoteric compound with emulsifying abilities as described herein. A surfactant operative herein is any conventional surfactant known to the art. The hydrophilic lipophilic balance (HLB) value of an inventive formulation independent of the natural essential oil is dictated by the desired balance between degreasing properties and aqueous washability. It should be understood that a second surfactant is often helpful in adjusting the inventive composition HLB value. It is further appreciated that the second surfactant is independently a nonionic, anionic, cationic, or amphoteric emulsifying compound.

Water often represents an essential component of a waterless hand sanitizer and is typically present from 5 to 70 total weight percent. In preparing a formulation, an oil phase containing organic solvent and other lipophilic components is mixed with a water phase. For instance, while fatty acids and low ethoxylation ratio nonionic surfactants are typically dissolved in an oil phase, amine soaps and high ethoxylation ratio nonionic surfactants are typically segregated in the water phase. Typically, the two phases are combined with heating to from 60° to 90° Celsius with constant stirring until a homogeneous smooth gel forms.

Another aspect of the present invention is a method of washing hands for an approximate predetermined period of time, which is determined based on the time required to achieve effective hand washing and may vary in different circumstances. The recommended time is approximately twenty seconds for purposes of achieving clean hands in ordinary hygiene circumstances. However, the present invention can be modified through changes in indicator concentrations to a different amount of time to indicate a longer or shorter time period of hand washing. Medical professionals, for example, may need to wash their hands for a longer period of time to achieve even cleaner hands. As can be seen, many variations of the method are possible.

Suitable optional additives to the compositions of the invention include, antibacterial agents, moisturizers, humectants, preservatives, fragrance, etc.

The term “humectant” is known and helps to retard the evaporation of water from the composition of the invention, thus avoiding premature drying during the application. Representative examples of humectants include, but are not limited to, polyhydroxy alkyls, such as glycerin, ethylene glycol, propylene glycol, diethylene glycol, polyethylene glycol, hydroxylated starches and mixtures of these materials. Any effective amount of humectant may be used. In one particular aspect, the humectant is glycerin.

Representative examples of preservatives include, but are not limited to, glutaraldehyde, bicyclic oxazolidones, hydroxybenzoic acid esters, 3-iodo-2-propynyl butyl carbamate, methyl p-hydroxybenzoate, and a biocide comprising 2-methyl-4-isothiazolin-3-one and 5-chloro-2-methyl-4-isothiazolin-3-one. The preservatives often serves as both a bactericide and a fungicide.

In particular, compositions of the invention include preservatives that are selected from, but not limited to, Liquid Germall Plus, iodopropynyl butyl carbamate, Germall II, diazolidinyl urea, Nuosept 95, bicyclic oxazolidines solution, manufactured by ISP (International Specialty Products), Wayne, N.J., Troysan 395, dihydroxy-dimethyl hydantoin, manufactured by Troy Chemical Corporation, Florham park, NJ and Kathon PFM, isothiazolinones, manufactured by Rohm & Haas Co., Philadelphia, Pa. Preservatives, when present in the compositions of the invention, are generally present in amounts from about 0.01% to about 6% by weight, in particular from about 0.05% to about 5% by weight, and particularly from about 0.1% to about 2.5% by weight. In one aspect, the preservative is one of Liquid Germall Plus, Tryosan 395 or Nuosept 95.

Representative fragrances include those pleasing to adults and children such as flowers, spices, candy, popcorn, fruit, bubble gum and the like. A fragrance, when present in the compositions of the invention, is generally present in amounts from about 0.1% to about 10% by weight of the total weight of the composition.

### EXAMPLES

Synthesis of Acid-Base Indicators:

### Example 1

### Synthesis of 3,3-bis-(4-hydroxy-3-isopropylphenyl)-1-(3H)-isobenzofuranone

A mixture of 2-isopropylphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-isopropylphenyl)-1-(3H)-isobenzofuranone in 96% yield.

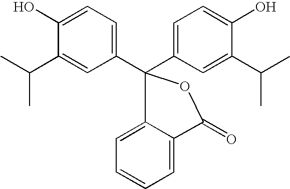

### Example 2

### Synthesis of 3,3-bis-(4-hydroxy-3,5-diisopropylphenyl)-1-(3H)-isobenzofuranone

A mixture of 2,6-diisopropylphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3,5-diisopropylphenyl)-1-(3H)-isobenzofuranone in 98% yield.

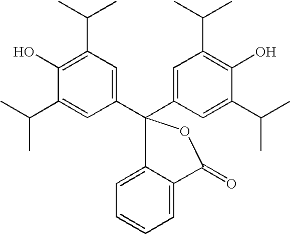

### Example 3

### Synthesis of 3,3-bis-(4-hydroxy-2-nitrophenyl)-1-(3H)-isobenzofuranone

A mixture of 3-nitrophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-2-nitrophenyl)-1-(3H)-isobenzofuranone in 81% yield.

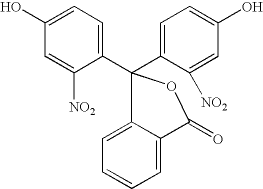

### Example 4

### Synthesis of 3,3-bis-(4-hydroxy-3-nitrophenyl)-1-(3H)-isobenzofuranone

A mixture of 2-nitrophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-nitrophenyl)-1-(3H)-isobenzofuranone in 89% yield.

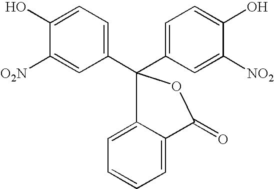

### Example 5

### Synthesis of 3,3-bis-[4-hydroxy-2-(N,N-diethylamino)phenyl]-1-(3H)-isobenzofuranone

A mixture of 3-(N,N-diethylamino)phenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-[4-hydroxy-2-(N,N-diethylamino)phenyl]-1-(3H)-isobenzofuranone in 93% yield.

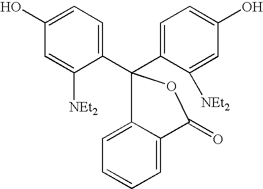

### Example 6

### Synthesis of 3,3-bis-(4-hydroxy-3-ethylphenyl)-1-(3H)-isobenzofuranone

A mixture of 2-ethylphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-ethylphenyl)-1-(3H)-isobenzofuranone in 92% yield.

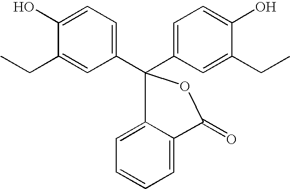

### Example 7

### Synthesis of 3,3-bis-(4-hydroxy-3-ethoxyphenyl)-1-(3H)-isobenzofuranone

A mixture of 2-ethoxyphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-ethoxyphenyl)-1-(3H)-isobenzofuranone in 85% yield.

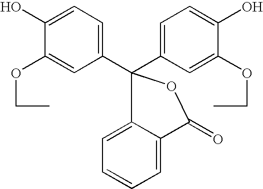

### Example 8

### Synthesis of 3,3-bis-(4-hydroxy-3-acetamidophenyl)-1-(3H)-isobenzofuranone

A mixture of 2-acetamidophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-acetamidophenyl)-1-(3H)-isobenzofuranone in 83% yield.

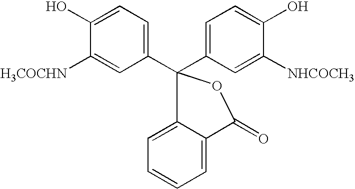

### Example 9

### Synthesis of 3,3-bis-(4-hydroxy-6-methyl-3-nitrophenyl)-1-(3H)-isobenzofuranone

A mixture of 5-methyl-2-nitrophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-6-methyl-3-nitrophenyl)-1-(3H)-isobenzofuranone in 81% yield.

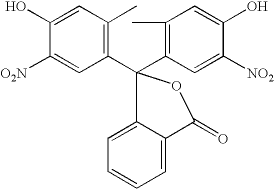

### Example 10

### Synthesis of 3,3-bis-(4-hydroxy-6-methyl-5-quinolin-1-yl)-1-(3H)-isobenzofuranone

A mixture of 8-hydroxyquinaldine (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-6-methyl-5-quinolin-1-yl)-1-(3H)-isobenzofuranone in 88% yield.

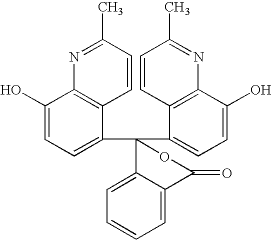

### Example 11

### Synthesis of 3,3-bis-(4-hydroxy-3-pyridin-1-yl)-1-(3H)-isobenzofuranone

A mixture of 2-hydroxypyridine (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-pyridin-1-yl)-1-(3H)-isobenzofuranone in 80% yield.

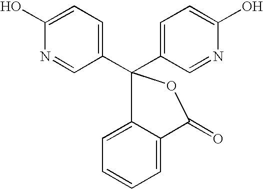

### Example 12

### Synthesis of 3,3-bis-(4-hydroxy-2-pyridin-1-yl)-1-(3H)-isobenzofuranone

A mixture of 3-hydroxypyridine (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-2-pyridin-1-yl)-1-(3H)-isobenzofuranone in 82% yield.

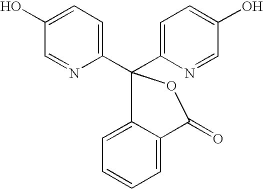

### Example 13

### Synthesis of 3,3-bis-(4-hydroxy-3-phenylphenyl)-1-(3H)-isobenzofuranone

A mixture of 2-phenylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3-phenylphenyl)-1-(3H)-isobenzofuranone as white crystals in 94% yield. ¹H-NMR (DMSO-d₆, 300 MHz): δ 9.89 (s, 2H, 2OH), 6.97-7.18 (m, 6H, aromatic), 7.26-7.47 (m, 10H, aromatic), 7.63-7.92 (m, 4H, aromatic) ppm. Mass spectra: m/z 470 (M⁺).

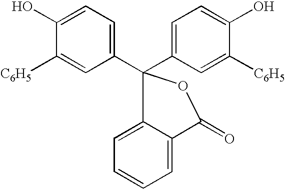

### Example 14

### Synthesis of 3,3-bis-(4-hydroxy-3,5-diisopropylphenyl)-1-(3 μl)-isobenzofuranone

A mixture of 2,6-diisopropylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,5-diisopropylphenyl)-1-(3H)-isobenzofuranone as white crystals in 89% yield. IR (KBr): 3506, 1734, 1609 cm⁻¹. ¹H-NMR (DMSO-d₆): δ 9.56 (s, 2H, 2OH), 1.02-1.05 (dd, 24H, 8CH₃), 3.22-3.31 (heptate, 4H, 4CH), 6.74-7.00 (m, 4H, aromatic), 7.59-7.92 (m, 4H, aromatic) ppm. Mass spectra: m/z 486 (M⁺).

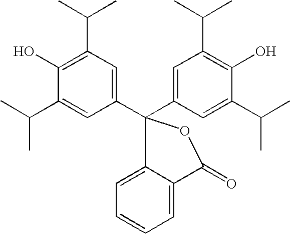

### Example 15

### Synthesis of 3,3-bis-(4-hydroxy-3,5-dimethoxyphenyl)-1-(3H)-isobenzofuranone

A mixture of 2,6-dimethoxyphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,5-dimethoxyphenyl)-1-(3H)-isobenzofuranone as white crystals in 84% yield. IR (KBr): 3388, 1769, 1606, 1369 cm⁻¹. ¹H-NMR (DMSO-d₆): δ 8.71 (s, 2H, 2OH), 3.66 (s, 12H, 40CH₃), 7.65-7.68 (m, 4H, aromatic), 7.83-7.96 (m, 4H, aromatic) ppm. Mass spectra: m/z 438 (M⁺).

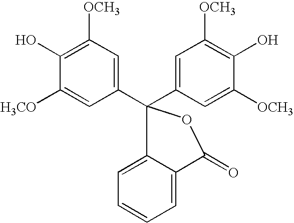

### Example 16

### Synthesis of 3,3-bis-(4-hydroxy-3,5-dimethylphenyl)-1-(3H)-isobenzofuranone

A mixture of 2,6-dimethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,5-dimethylphenyl)-1-(3H)-isobenzofuranone as white crystals in 91% yield. IR (KBr): 3582, 3386, 1746, 1605 cm⁻. ¹H-NMR (DMSO-d₆): δ 8.45 (s, 2H, 2OH), 2.10 (s, 12H, 4CH₃), 7.58-7.63 (m, 4H, aromatic), 7.78-7.87 (m, 4H, aromatic) ppm. Mass spectra: m/z 374 (M⁺).

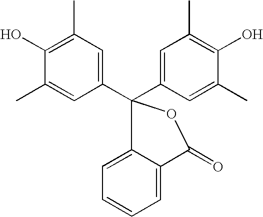

### Example 17

### Synthesis of 3,3-bis-(4-hydroxy-3,6-diimethylphenyl)-1-(3H)-isobenzofuranone

A mixture of 2,5-dimethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,6-diimethylphenyl)-1-(3H)-isobenzofuranone as pale yellow crystals in 85% yield. IR (KBr): 3393, 1729, 1611 cm⁻¹. ¹H-NMR (DMSO-d₆): δ 9.40 (s, 2H, 2OH), 1.95 (s, 12H, 4CH₃), 6.59-6.63 (m, 4H, aromatic), 7.46-7.91 (m, 4H, aromatic) ppm. Mass spectra: m/z 374 (M⁺).

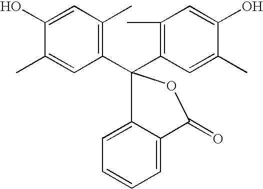

### Example 18

### Synthesis of 3,3-bis-(4-hydroxy-3-ethylphenyl)-1-(3H)-isobenzofuranone

A mixture of 2-ethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from ethyl acetate:petroleum ether (1:1) with charcoal treatment furnished pure 3,3-bis-(4-hydroxy-3-ethylphenyl)-1-(3H)-isobenzofuranone as white crystals in 81% yield. IR (KBr): 3389, 1783, 1718, 1605 cm⁻. ¹H-NMR (DMSO-d₆): δ 9.54 (s, 2H, 2OH), 2.43-2.50 (q, 4H, 2CH₂), 1.00-1.05 (t, 6H, 2CH₃), 6.74-6.96 (m, 6H, aromatic), 7.57-7.89 (m, 4H, aromatic) ppm. Mass spectra: m/z 374 (M⁺).

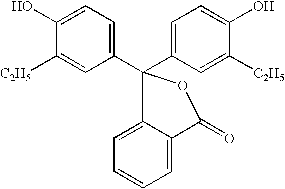

### Example 19

### Synthesis of 3,3-bis-(4-hydroxy-3-isopropylphenyl)-1-(3H)-isobenzofuranone

A mixture of 2-isopropylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol:water (1:1) with charcoal treatment furnished pure 33,3-bis-(4-hydroxy-3-isopropylphenyl)-1-(3H)-isobenzofuranone as white crystals in 83% yield. IR (KBr): 3383, 1733, 1608 cm⁻¹. ¹H-NMR (DMSO-d₆): δ 9.57 (s, 2H, 2OH), 1.05-1.07 (dd, 12H, 4CH₃), 3.11-3.18 (heptate, 2H, 2CH), 6.75-7.01 (m, 6H, aromatic), 7.59-7.90 (m, 4H, aromatic) ppm. Mass spectra: m/z 402 (M⁺).

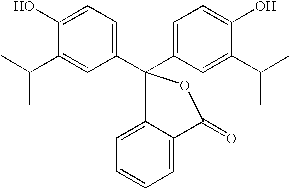

### Example 20

### Synthesis of 3,3-bis-(4-hydroxy-3-methoxyphenyl)-1-(3H)-isobenzofuranone

A mixture of 2-methoxyphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3-methoxyphenyl)-1-(3H)-isobenzofuranone as white crystals in 79% yield. IR (KBr): 3517, 1747, 1701, 1279 cm⁻¹. ¹H-NMR (DMSO-d₆): δ 9.27 (s, 2H, 2OH), 3.66 (s, 6H, 2OCH₃), 6.65-6.78 (m, 6H, aromatic), 7.61-7.90 (m, 4H, aromatic) ppm. Mass spectra: m/z 378 (M⁺).

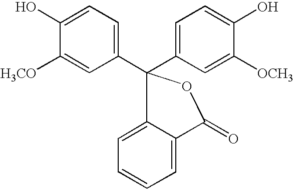

### Example 21

### Synthesis of 3,3-bis-(4-hydroxy-2,3,5-trimethylphenyl)-1-(3H)-isobenzofuranone

A mixture of 2,3,6-trimethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-2,3,5-trimethylphenyl)-1-(3H)-isobenzofuranone as white crystals in 73% yield. IR (KBr): 3510, 3390, 1746, 1609, cm⁻¹. ¹H-NMR (DMSO-d₆): δ 9.44 (s, 2H, 2OH), 2.05 (s, 18H, 6CH₃), 6.55 (s, 2H, aromatic), 7.46-7.90 (m, 4H, aromatic) ppm. Mass spectra: m/z 402 (M⁺).

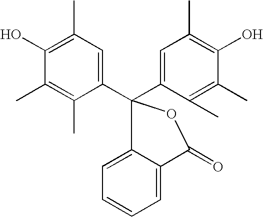

### Example 22

### Synthesis of 3,3-bis-(4-hydroxy-3-sec-butylphenyl)-1-(3H)-isobenzofuranone

A mixture of 2-sec-butylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3-sec-butylphenyl)-1-(3H)-isobenzofuranone as white crystals in 77% yield. IR (KBr): 3400, 1722, 1607 cm⁻¹. ¹H-NMR (DMSO-d₆): δ 9.50 (s, 2H, 2OH), 0.80 (t, 6H, 2CH₃), 1.35-1.39 (p, 4H, 2CH₂), 1.22 (d, 6H, 2CH₃), 2.89-2.97 (sextate, 2H, 2CH), 6.73-6.93 (m, 6H, aromatic), 7.59-7.90 (m, 4H, aromatic) ppm. Mass spectra: m/z 430 (M⁺).

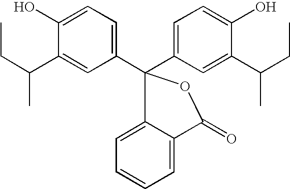

### Example 23

### Synthesis of 3,3-bis-(4-hydroxy-3-nitrophenyl)-1-(3H)-isobenzofuranone

A mixture of phenolphthalein (0.062 mol) in acetic acid (290 mL) was stirred at 15° C. Concentrated nitric acid (0.136 mol, 65%) in acetic acid (10 mL) was slowly added to stirring mixture at 15° C. The reaction mixture was further stirred for 6 hours at room temperature and added to ice-water mixture when the yellow colored product precipitated. The product was filtered, thoroughly washed with water and dried. Recrystallization from ethanol with charcoal treatment furnished pure 3,3-bis-(4-hydroxy-3-nitrophenyl)-1-(3H)-isobenzofuranone as pale yellow crystals in 78% yield. IR (KBr): 3262, 1766, 1627, 1538, 1423 cm⁻¹.

¹H-NMR (DMSO-d₆): δ 9.67 (s, 2H, 2OH), 6.71-7.16 (m, 6H, aromatic), 7.46-7.98 (m, 4H, aromatic) ppm. Mass spectra: m/z 408 (M⁺). 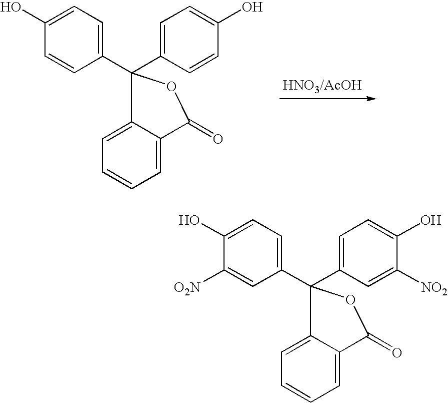
Synthesis of Disodium Salts of Acid-Base Indicators for Water Based Systems:

### Example 1

### Synthesis of disodium salt of 3,3-bis-(4-hydroxy-3-isopropylphenyl)-1-(3H)-isobenzofuranone

A mixture of 3,3-bis-(4-hydroxy-3-isopropylphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 98% yield.

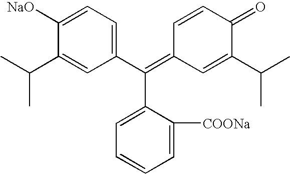

### Example 2

### Synthesis of disodium salt of 3,3-bis-(4-hydroxy-3,5-diisopropylphenyl)-1-(3H)-isobenzofuranone

A mixture of 3,3-bis-(4-hydroxy-3,5-diisopropylphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 94% yield.

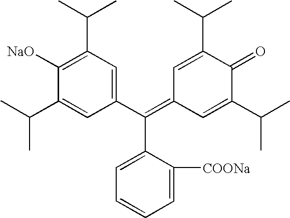

### Example 3

### Synthesis of disodium salt of 3,3-bis-(4-hydroxy-2-nitrophenyl)-1-(3H)-isobenzofuranone

A mixture of 3,3-bis-(4-hydroxy-2-nitrophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 88% yield.

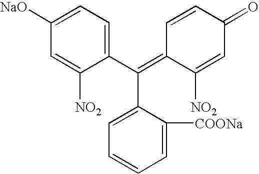

### Example 4

### Synthesis of disodium salt of 3,3-bis-(4-hydroxy-3-nitrophenyl)-1-(3H)-isobenzofuranone

A mixture of 3,3-bis-(4-hydroxy-3-nitrophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 91% yield.

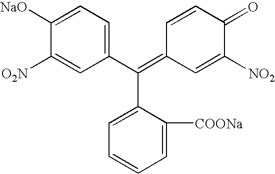

### Example 5

### Synthesis of disodium salt of 3,3-bis-[4-hydroxy-2-(N,N-diethylamino)phenyl]-1-(3H)-isobenzofuranone

A mixture of 3,3-bis-[4-hydroxy-2-(N,N-diethylamino)phenyl]-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure, disodium salt in 89% yield.

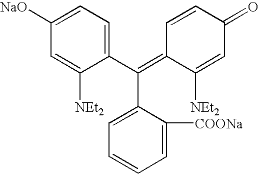

### Example 6

### Synthesis of disodium salt of 3,3-bis-(4-hydroxy-3-ethylphenyl)-1-(3H)-isobenzofuranone

A mixture of 3,3-bis-(4-hydroxy-3-ethylphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 97% yield.

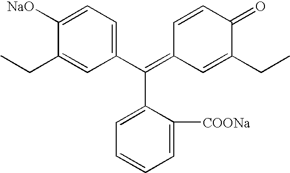

### Example 7

### Synthesis of disodium salt of 3,3-bis-(4-hydroxy-3-ethoxyphenyl)-1-(3H)-isobenzofuranone

A mixture of 3,3-bis-(4-hydroxy-3-ethoxyphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 94% yield.

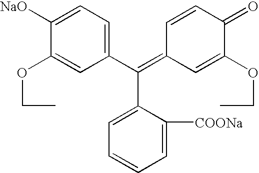

### Example 8

### Synthesis of disodium salt of 3,3-bis-(4-hydroxy-3-acetamidophenyl)-1-(3H)-isobenzofuranone

A mixture of 3,3-bis-(4-hydroxy-3-acetamidophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 92% yield.

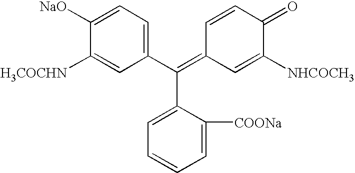

### Example 9

### Synthesis of disodium salt of 3,3-bis-(4-hydroxy-6-methyl-3-nitrophenyl)-1-(3H)-isobenzofuranone

A mixture of 3,3-bis-(4-hydroxy-6-methyl-3-nitrophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 97% yield.

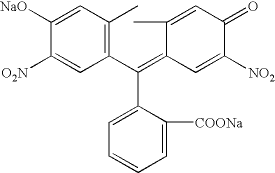

### Example 10

### Synthesis of disodium salt of 3,3-bis-(4-hydroxy-6-methyl-5-quinolin-1-yl)-1-(3H)-isobenzofuranone

A mixture of 3,3-bis-(4-hydroxy-6-methyl-5-quinolin-1-yl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 94% yield.

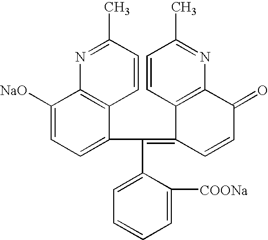

### Example 11

### Synthesis of disodium salt of 3,3-bis-(4-hydroxy-3-pyridin-1-yl)-1-(3H)-isobenzofuranone

A mixture of 3,3-bis-(4-hydroxy-3-pyridin-1-yl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 81% yield.

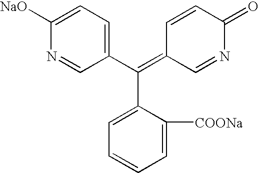

### Example 12

### Synthesis of disodium salt of 3,3-bis-(4-hydroxy-2-pyridin-1-yl)-1-(3H)-isobenzofuranone

A mixture of 3,3-bis-(4-hydroxy-2-pyridin-1-yl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 84% yield.

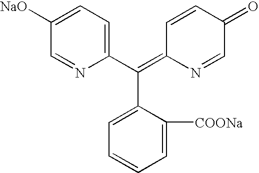

### Example 13

### Synthesis of disodium salt of 3,3-bis-(4-hydroxy-3-phenylphenyl)-1-(3H)-isobenzofuranone

A mixture of 3,3-bis-(4-hydroxy-3-phenylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 96% yield. ¹H-NMR (DMSO-d₆, 300 MHz): δ 6.25-6.74 (m, 6H, aromatic), 6.88-7.45 (m, 10H, aromatic), 7.53-7.84 (m, 4H, aromatic) ppm. Mass spectra: m/z 514 (M⁺).

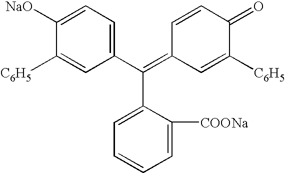

### Example 14

### Synthesis of disodium salt of 3,3-bis-(4-hydroxy-3,5-diisopropylphenyl)-1-(3H)-isobenzofuranone

A mixture of 3,3-bis-(4-hydroxy-3,5-diisopropylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 92% yield. ¹H-NMR (DMSO-d₆): δ 1.00-1.21 (dd, 24H, 8CH₃), 3.06-3.36 (heptate, 4H, 4CH), 6.74-6.96 (m, 4H, aromatic), 7.05-7.83 (m, 4H, aromatic) ppm. Mass spectra: m/z 530 (M⁺).

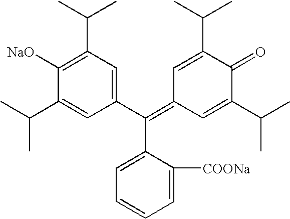

### Example 15

### Synthesis of disodium salt of 3,3-bis-(4-hydroxy-3,5-dimethoxyphenyl)-1-(3H)-isobenzofuranone

A mixture of 3,3-bis-(4-hydroxy-3,5-dimethoxyphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 90% yield. ¹H-NMR (DMSO-d₆): δ 3.61 (s, 12H, 4OCH₃), 6.45-6.52 (m, 4H, aromatic), 7.04-7.78 (m, 4H, aromatic) ppm. Mass spectra: m/z 482 (M⁺).

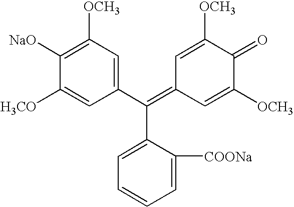

### Example 16

### Synthesis of disodium salt of 3,3-bis-(4-hydroxy-3,5-dimethylphenyl)-1-(3H)-isobenzofuranone

A mixture of 3,3-bis-(4-hydroxy-3,5-dimethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 95% yield. ¹H-NMR (DMSO-d₆): δ 2.11 (s, 12H, 4CH₃), 6.81-6.87 (m, 4H, aromatic), 7.23-7.84 (m, 4H, aromatic) ppm. Mass spectra: m/z 418 (M⁺).

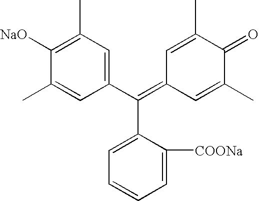

### Example 17

### Synthesis of disodium salt of 3,3-bis-(4-hydroxy-3,6-diimethylphenyl)-1-(3H)-isobenzofuranone

A mixture of 3,3-bis-(4-hydroxy-3,6-diimethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 88% yield. ¹H-NMR (DMSO-d₆): δ 2.01 (s, 12H, 4CH₃), 6.04-6.82 (m, 4H, aromatic), 7.10-7.72 (m, 4H, aromatic) ppm. Mass spectra: m/z 418 (M⁺).

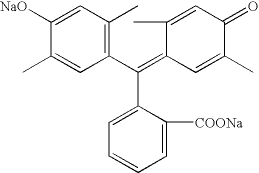

### Example 18

### Synthesis of disodium salt of 3,3-bis-(4-hydroxy-3-ethylphenyl)-1-(3H)-isobenzofuranone

A mixture of 3,3-bis-(4-hydroxy-3-ethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 86% yield. ¹H-NMR (DMSO-d₆): δ 2.30-2.51 (q, 4H, 2CH₂), 1.00-1.10 (t, 6H, 2CH₃), 6.20-6.75 (m, 6H, aromatic), 7.12-7.84 (m, 4H, aromatic) ppm. Mass spectra: m/z 418 (M⁺).

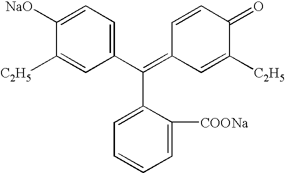

### Example 19

### Synthesis of disodium salt of 3,3-bis-(4-hydroxy-3-isopropylphenyl)-1-(3H)-isobenzofuranone

A mixture of 3,3-bis-(4-hydroxy-3-isopropylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 81% yield. ¹H-NMR (DMSO-d₆): δ 1.02-1.14 (dd, 12H, 4CH₃), 3.12-3.45 (heptate, 2H, 2CH), 6.32-6.76 (m, 6H, aromatic), 7.30-7.83 (m, 4H, aromatic) ppm. Mass spectra: m/z 446 (M⁺).

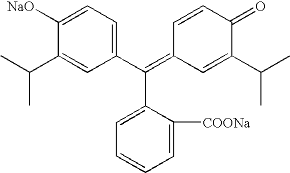

### Example 20

### Synthesis of disodium salt of 3,3-bis-(4-hydroxy-3-methoxyphenyl)-1-(3H)-isobenzofuranone

A mixture of 3,3-bis-(4-hydroxy-3-methoxyphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 88% yield.

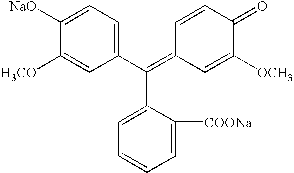

### Example 21

### Synthesis of disodium salt of 3,3-bis-(4-hydroxy-2,3,5-trimethylphenyl)-1-(3H)-isobenzofuranone

A mixture of 3,3-bis-(4-hydroxy-2,3,5-trimethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 80% yield.

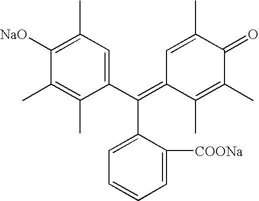

### Example 22

### Synthesis of disodium salt of 3,3-bis-(4-hydroxy-3-sec-butylphenyl)-1-(3H)-isobenzofuranone

A mixture of 3,3-bis-(4-hydroxy-3-sec-butylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 82% yield. 1H-NMR (DMSO-d₆): δ 0.80 (t, 6H, 2CH₃), 1.29-1.37 (p, 4H, 2CH₂), 1.20 (d, 6H, 2CH₃), 2.88-2.96 (sextate, 2H, 2CH), 6.08-6.75 (m, 6H, aromatic), 7.37-7.81 (m, 4H, aromatic) ppm.

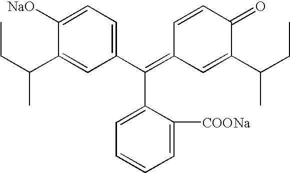

### Example 23

### Synthesis of disodium salt of 3,3-bis-(4-hydroxy-3-nitrophenyl)-1-(3H)-isobenzofuranone

A mixture of 3,3-bis-(4-hydroxy-3-nitrophenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 92% yield. 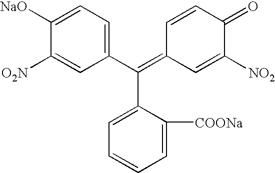
Synthesis of Phenol and Hydrazine Acid-Base Indicators

### Example 1

### Synthesis of sodium salt of 5-methyl-2-nitrophenol

A mixture of 5-methyl-2-nitrophenol (0.1M) in ethanol (25 mL, 85%) was stirred followed by addition of sodium hydroxide (0.1M) in ethanol (25 mL, 85%). The reaction mixture was stirred at room temperature for 2 hours. The separated golden yellow solid was filtered, washed with ethanol and dried. Recrystallization from ethanol furnished pure sodium salt in 96% yield.

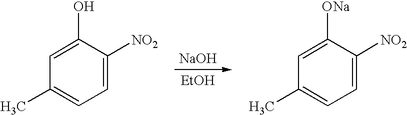

### Example 2

### Synthesis of p-nitrobenzhydrazide

A mixture of ethyl p-nitrobenzoate (0.1M), hydrazine hydrate (0.11M) in ethanol (100 mL) was stirred at room temperature for 2 hours. The separated pale yellow solid was filtered, washed with ethanol and dried. Recrystallization from ethanol furnished pure hydrazide in 88% yield.

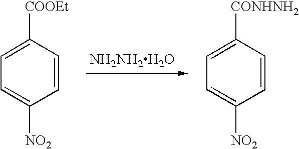

### Example 3

### Synthesis of Hydrazide

A mixture of ethyl salicylate (0.1M), 2,4-dinitrophenylhydrazine (0.1M) in ethanol (150 mL) was stirred at room temperature for 2 hours. The separated orange solid was filtered, washed with ethanol and dried. Recrystallization from ethanol furnished pure hydrazide in 94% yield.

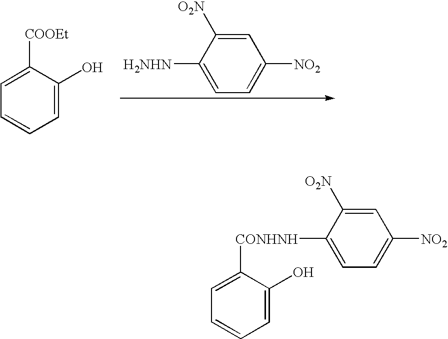

### Example 4

### Synthesis of Sodium Salt of Hydrazide

A mixture of hydrazide (0.1M) in ethanol (25 mL) was stirred followed by addition of sodium ethoxide (0.1M) in ethanol (25 mL). The reaction mixture was stirred and refluxed at for 4 hours, cooled to room temperature. The solvent was evaporated on rotary evaporator, isolated the crude yellow product and dried. Recrystallization from ethanol furnished pure sodium salt in 92% yield.

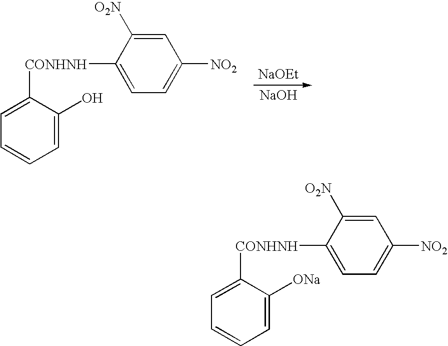

### Example 5

### Synthesis of Hydrazide

A mixture of ethyl salicylate (0.1M), 4-nitrophenylhydrazine (0.1M) in ethanol (150 mL) was stirred at room temperature for 2 hours. The separated yellow solid was filtered, washed with ethanol and dried. Recrystallization from ethanol furnished pure hydrazide in 89% yield.

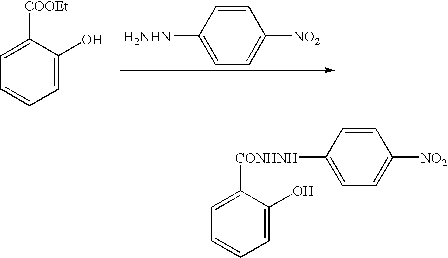

### Example 6

### Synthesis of Sodium Salt of Hydrazide

A mixture of hydrazide (0.1M) in ethanol (25 mL) was stirred followed by addition of sodium ethoxide (0.1M) in ethanol (25 mL). The reaction mixture was stirred and refluxed at for 4 hours, cooled to room temperature. The solvent was evaporated on rotary evaporator, isolated the crude yellow product and dried. Recrystallization from ethanol furnished pure sodium salt in 84% yield.

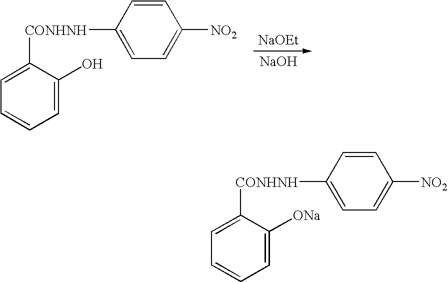

Soap Solution

### Example 1

| Chemical Component | Weight in grams |
|---|---|
| 5% NaOH Solution | 6.74 |
| 20% SLS Solution | 16 |
| Deionized water | 75.44 |
| Thymolphthalein | 1.81 |

Procedure: Combine 5% NaOH solution, water and SLS. Add indicator and stir vigorously overnight.

Results: A blue hand soap that turns clear after 10 seconds of rubbing hands together.

### Example 2

| Chemical Component | Weight in grams |
|---|---|
| 5% NaOH Solution | 2.05 |
| 20% SLS Solution | 16 |
| Deionized water | 81.51 |
| O-cresolphthalein | .44 |

Procedure: Combine 5% NaOH solution, water and SLS. Add indicator and stir vigorously overnight.

Results: A pink hand soap that turns clear after 10 seconds of rubbing hands together.

### Example 3

| Chemical Component | Weight in grams |
|---|---|
| 5% NaOH Solution | 3.11 |
| 20% SLS Solution | 16 |
| Deionized water | 79.97 |
| Phenylphenolphthalein | .92 |

Procedure: Combine 5% NaOH solution, water and SLS. Add indicator and stir vigorously overnight.

Results: A pink hand soap that turns clear after 10 seconds of rubbing hands together.

### Example 4

| Chemical Component | Weight in grams |
|---|---|
| 5% NaOH Solution | 7.0 |
| 20% SLS Solution | 16 |
| Deionized water | 74 |
| Thymolphthalein | 3.0 |

Procedure: Combine 5% NaOH solution, water and SLS. Add indicator and stir vigorously overnight.

Results: A blue hand soap that turns clear after 15 seconds of rubbing hands together.

### Example 5

| Chemical Component | Weight in grams |
|---|---|
| 5% NaOH Solution | 2.50 |
| 20% SLS Solution | 16 |
| Deionized water | 79.5 |
| O-cresolphthalein | 2 |

Procedure: Combine 5% NaOH solution, water and SLS. Add indicator and stir vigorously overnight.

Results: A pink hand soap that turns clear after 20 seconds of rubbing hands together.

### Example 6

| Chemical Component | Weight in grams |
|---|---|
| 5% NaOH Solution | 3.11 |
| 20% SLS Solution | 16 |
| Deionized water | 79.97 |
| 2-Phenylphenolphthalein | .92 |

Procedure: In a 3-neck 100 ml RB flask with reflux condenser and thermometer add 40 g methanesulfonic acid and 22.64 g 2-phenylphenol. Add slowly with stirring 11 g phthalic anhydride. Stir and heat at 90° C. for 12 hours. Add the hot reaction mixture to ice/water mixture. When the product precipitates, collect by filtration, wash thoroughly with water and dry at 50° C. overnight. The resulting material is 2-Phenylphenolphthalein indicator. Combine 5% NaOH solution, water and SLS. Add indicator and stir vigorously overnight.

Results: A purple hand soap that turns clear after 20 seconds of rubbing hands together.

### Example 7

| Chemical Component | Weight in grams |
|---|---|
| 5% NaOH Solution | 3.11 |
| 20% SLS Solution | 16 |
| Deionized water | 79.97 |
| 2,6-dimethylphenolphthalein | .92 |

Procedure: In a 3-neck 100 mL RB flask fitted with reflux condenser, thermometer and glass stopper was added methane sulfonic acid (40 g, 0.416 mol) followed by addition of 2,6-dimethylphenol (16.24 g, 0.133 mol). Phthalic anhydride (11 g, 0.074 mol) was slowly added under stirring. The reaction mixture was stirred and heated at 90° C. for 12 hours. The hot reaction mixture was added to ice-water mixture when the product precipitated. The product was filtered thru buchner funnel, thoroughly washed with water and dried in oven overnight at 50° C. The resulting material is 2,6-dimethylphenolphthalein indicator. Combine 5% NaOH solution, water and SLS. Add indicator and stir vigorously overnight.

Results: A purple hand soap that turns clear after 30 seconds of rubbing hands together.

Liquid Soap Examples:

### Example 1

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| Thymolphthalein | 0.5 |
| Sodium hydroxide | 0.09 |
| Deionized water | 81.90 |
| Liquid Germall Plus | 0.1 |

Amphosol HCA, Steol CS-330, Ninol 40-CO and glycerin were stirred at room temperature for 30 minutes till it became homogeneous. Thymolphthalein, sodium hydroxide, deionized water, sodium chloride and liquid germall plus were added and mixture was further stirred for 3 hours at room temperature.

Color of the solution: Blue

Color change: Blue to colorless

### Example 2

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| o-Cresolphthalein | 0.5 |
| Sodium hydroxide | 0.11 |
| Deionized water | 81.88 |
| Liquid Germall Plus | 0.1 |

Amphosol HCA, Steol CS-330, Ninol 40-CO and glycerin were stirred at room temperature for 30 minutes till it became homogeneous. o-Cresolphthalein, sodium hydroxide, deionized water, sodium chloride and liquid germall plus were added and mixture was further stirred for 3 hours at room temperature.

Color of the solution: Magenta

Color change: Magenta to colorless

### Example 3

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3-phenylphenyl)- | 0.5 |
| 1-(3H)-isobenzofuranone | |
| Sodium hydroxide | 0.08 |
| Deionized water | 81.91 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3-phenylphenyl)-1-(3H)-isobenzofuranone, sodium hydroxide, deionized water, sodium chloride and liquid germall plus were added and mixture was further stirred for 3 hours at room temperature.

Color of the solution: Purple

Color change: Purple to colorless

### Example 4

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3,5-diisopropylphenyl)- | 0.5 |
| 1-(3H)-isobenzofuranone | |
| Sodium hydroxide | 0.08 |
| Deionized water | 81.91 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3,5-diisopropylphenyl)-1-(3H)-isobenzofuranone, sodium hydroxide, deionized water, sodium chloride and liquid germall plus were added and mixture was further stirred for 3 hours at room temperature.

Color of the solution: Violet

Color change: Violet to colorless

### Example 5

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3,5-dimethoxyphenyl)- | 0.5 |
| 1-(3H)-isobenzofuranone | |
| Sodium hydroxide | 0.09 |
| Deionized water | 81.90 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3,5-dimethoxyphenyl)-1-(3H)-isobenzofuranone, sodium hydroxide, deionized water, sodium chloride and liquid germall plus were added and mixture was further stirred for 3 hours at room temperature.

Color of the solution: Teal

Color change: Teal to colorless

### Example 6

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3,5-dimethylphenyl)- | 0.5 |
| 1-(3H)-isobenzofuranone | |
| Sodium hydroxide | 0.1 |
| Deionized water | 81.89 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3,5-dimethylphenyl)-1-(3H)-isobenzofuranone, sodium hydroxide, deionized water, sodium chloride and liquid germall plus were added and mixture was further stirred for 3 hours at room temperature.

Color of the solution: Purple

Color change: Purple to colorless

### Example 7

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3,6-diimethylphenyl)- | 0.5 |
| 1-(3H)-isobenzofuranone | |
| Sodium hydroxide | 0.1 |
| Deionized water | 81.89 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3,6-diimethylphenyl)-1-(3H)-isobenzofuranone, sodium hydroxide, deionized water, sodium chloride and liquid germall plus were added and mixture was further stirred for 3 hours at room temperature.

Color of the solution: Blue

Color change: Blue to colorless

### Example 8

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3-ethylphenyl)- | 0.5 |
| 1-(3H)-isobenzofuranone | |
| Sodium hydroxide | 0.1 |
| Deionized water | 81.89 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3-ethylphenyl)-1-(3H)-isobenzofuranone, sodium hydroxide, deionized water, sodium chloride and liquid germall plus were added and mixture was further stirred for 3 hours at room temperature.

Color of the solution: Magenta

Color change: Magenta to colorless

### Example 9

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-hydroxy-3-isopropylphenyl)- | 0.5 |
| 1-(3H)-isobenzofuranone | |
| Sodium hydroxide | 0.09 |
| Deionized water | 81.90 |
| Liquid Germall Plus | 0.1 |

Amphosol HCA, Steol CS-330, Ninol 40-CO and glycerin were stirred at room temperature for 30 minutes till it became homogeneous. 3,3-bis-(4-hydroxy-3-isopropylphenyl)-1-(3H)-isobenzofuranone, sodium hydroxide, deionized water, sodium chloride and liquid germall plus were added and mixture was further stirred for 3 hours at room temperature.

Color of the solution: Pink

Color change: Pink to colorless

### Example 10

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3-methoxyphenyl)- | 0.5 |
| 1-(3H)-isobenzofuranone | |
| Sodium hydroxide | 0.1 |
| Deionized water | 81.89 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3-methoxyphenyl)-1-(3H)-isobenzofuranone, sodium hydroxide, deionized water, sodium chloride and liquid germall plus were added and mixture was further stirred for 3 hours at room temperature.

Color of the solution: Blue

Color change: Blue to colorless

### Example 11

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-2,3,5-trimethylphenyl)- | 0.5 |
| 1-(3H)-isobenzofuranone | |
| Sodium hydroxide | 0.09 |
| Deionized water | 81.90 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-2,3,5-trimethylphenyl)-1-(3H)-isobenzofuranone, sodium hydroxide, deionized water, sodium chloride and liquid germall plus were added and mixture was further stirred for 3 hours at room temperature.

Color of the solution: Teal

Color change: Teal to colorless

### Example 12

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| Bromo-thymolphthalein | 0.5 |
| Sodium hydroxide | 0.08 |
| Deionized water | 81.91 |
| Liquid Germall Plus | 0.1 |

Amphosol HCA, Steol CS-330, Ninol 40-CO and glycerin were stirred at room temperature for 30 minutes till it became homogeneous. Bromo-thymolphthalein, sodium hydroxide, deionized water, sodium chloride and liquid germall plus was added and mixture was further stirred for 3 hours at room temperature.

Color of the solution: Teal

Color change: Teal to colorless

### Example 13

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| Bromo-o-cresolphthalein | 0.5 |
| Sodium hydroxide | 0.1 |
| Deionized water | 81.89 |
| Liquid Germall Plus | 0.1 |

Amphosol HCA, Steol CS-330, Ninol 40-CO and glycerin were stirred at room temperature for 30 minutes till it became homogeneous. Bromo-o-cresolphthalein, sodium hydroxide, deionized water, sodium chloride and liquid germall plus was added and mixture was further stirred for 3 hours at room temperature.

Color of the solution: Purple

Color change: Purple to colorless

### Example 14

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3-sec-butylphenyl)- | 0.5 |
| 1-(3H)-isobenzofuranone | |
| Sodium hydroxide | 0.08 |
| Deionized water | 81.91 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3-sec-butylphenyl)-1-(3H)-isobenzofuranone, sodium hydroxide, deionized water, sodium chloride and liquid germall plus were added and mixture was further stirred for 3 hours at room temperature.

Color of the solution: Pink

Color change: Pink to colorless

### Example 15

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| 3,3-bis-(4-Hydroxy-3-nitrophenyl)- | 0.5 |
| 1-(3H)-isobenzofuranone | |
| Sodium hydroxide | 0.08 |
| Deionized water | 81.91 |
| Liquid Germall Plus | 0.1 |

Amphosol HCA, Steol CS-330, Ninol 40-CO and glycerin were stirred at room temperature for 30 minutes till it became homogeneous. 3,3-bis-(4-Hydroxy-3-nitrophenyl)-1-(3H)-isobenzofuranone, sodium hydroxide, deionized water, sodium chloride and liquid germall plus were added and mixture was further stirred for 3 hours at room temperature.

Color of the solution: Yellow

Color change: Yellow to colorless

### Example 16

| Chemical Component | Weight in grams |
|---|---|
| Amphosol HCA | 4.0 |
| Steol CS-330 | 10.0 |
| Ninol 40-CO | 1.5 |
| Glycerin | 0.5 |
| Sodium chloride | 1.41 |
| m-Nitrophenol | 0.5 |
| Sodium hydroxide | 0.08 |
| Deionized water | 81.91 |
| Liquid Germall Plus | 0.1 |

Amphosol HCA, Steol CS-330, Ninol 40-CO and glycerin were stirred at room temperature for 30 minutes till it became homogeneous. m-Nitrophenol, sodium hydroxide, deionized water, sodium chloride and liquid germall plus were added and mixture was further stirred for 3 hours at room temperature.

Color of the solution: Golden yellow

Color change: Golden yellow to colorless

### Example 17

| Chemical Component | Weight in grams |
|---|---|
| Commercial Liquid Soap^{a,b,c,d,e,f,g,h} | 50 |
| Dyeⁱ | 0.5 |
| Sodium hydroxide | 2 equivalent |
| Commercial Liquid Soap^{a}: Commercial Liquid Soaps used include but not limited to Dial Complete Foaming Hand Wash (Dial), Softsoap (Colgate), Antibacterial Clean & Smooth | |
| # (Ecolab), Super Clean & Smooth (Ecolab), Digiklenz Antibacterial Hand Soap (Ecolab), Nexcare (3M), Purell (GoJo) | |
| Commercial Liquid Soap^{b}: Dial Complete ingredients include but not limited to triclosan, water, sodium xylenesulfonate, dipropylene glycol, ammonium lauryl sulfate, | |
| # cocamidopropyl betaine, fragrance (parfum), disodium phosphate, citric acid, Red 4, Yellow 5. | |
| Commercial Liquid Soap^{c}: Softsoap ingredients include but not limited to water, sodium C14-C16 olefin sulfonate, lauramide DEA, glycol stearate, sodium chloride, cocamidopropyl betaine, citric acid, fragrance, DMDM hydantoin, polyquatemium-7, aloe barbadensis | |
| # leaf juice, tetrasodium EDTA, glycerin, hydrolyzed silk | |
| Commercial Liquid Soap^{d}: Antibacterial Clean & Smooth ingredients include but not limited to sulfuric acid, mono C10-C16 alkyl esters, water, sodium salts d-glucopyranose, | |
| # oligomeric C10-C16 alkyl glycosides poly(oxy-l,2-ethandilyl), alfa-sulfo-omega-hydroxy C10-C16 alkyl ether sodium salts, sodium chloride | |
| Commercial Liquid Soap^{e}: Super Clean & Smooth ingredients include but not limited to surfactant blend, triclosan | |
| Commercial Liquid Soap^{f}: Digiklenz Antibacterial Hand Soap ingredients include but not limited to ethanol, poly(oxy-l,2-ethandilyl), alfa-sulfo-omega-(dodecyloxy)-, sodium salts, methylpentane-2,4-diols, 1-propanaminium, 3,3’,3”-[phosphinylidynetris(oxy)]- | |
| # tris[n-(3-aminopropyl)-2-hydroxy-N,N-dimethyl-, N,N’,N”-tri-C6-C18 acyl derivatives trichlorides. | |
| Commercial Liquid Soap^{g}: Nexcare ingredients include but not limited to ethyl alcohol, beheneth-10, behenyl alcohol, C20-C40 pereth-24, cetyl palmitate, diisopropyl dimmer dilinolate, dimethicone, glycerin, | |
| # polyethylene glycol, squalane, water | |

Commercial Liquid Soap^{h}: Purell ingredients include but not limited to ethyl alcohol, water, glycerin, isopropyl myristate, propylene glycol, tocopheryl acetate, aminomethyl propanol, carbomer, fragrance (parfum) beheneth-10, behenyl alcohol, C20-C40 pereth-24, cetyl palmitate, diisopropyl dimmer dilinolate, dimethicone, glycerin, polyethylene glycol, squalane, water
Dyeⁱ: All the above dyes were used.

In a beaker containing commercial liquid soap was added dye and sodium hydroxide. The reaction mixture was further stirred for 2 hours at room temperature.

Test Procedure:

Colored liquid soap from any of the above examples was taken in both the hands and rubbed for 30 seconds to 1 minute, the respective color disappears.

Shampoo Examples:

### Example 1

| Chemical Component | Weight in grams |
|---|---|
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| Thymolphthalein | 0.5 |
| Sodium hydroxide | 1.0 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. Thymolphthalein, sodium hydroxide, deionized water, sodium chloride and liquid germall plus were added and mixture was further stirred for 4 hours at room temperature.

Color of the solution: Blue

Color change: Blue to colorless

### Example 2

| Chemical Component | Weight in grams |
|---|---|
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| o-Cresolphthalein | 0.5 |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. o-Cresolphthalein, sodium hydroxide, deionized water, sodium chloride and liquid germall plus were added and mixture was further stirred for 4 hours at room temperature.

Color of the solution: Magenta

Color change: Magenta to colorless

### Example 3

| Chemical Component | Weight in grams |
|---|---|
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3-phenylphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3-phenylphenyl)-1-(3H)-isobenzofuranone, sodium hydroxide, deionized water, sodium chloride and liquid germall plus were added and mixture was further stirred for 4 hours at room temperature.

Color of the solution: Purple

Color change: Purple to colorless

### Example 4

| Chemical Component | Weight in grams |
|---|---|
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3,5-diisopropylphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3,5-diisopropylphenyl)-1-(3H)-isobenzofuranone, sodium hydroxide, deionized water, sodium chloride and liquid germall plus were added and mixture was further stirred for 4 hours at room temperature.

Color of the solution: Violet

Color change: Violet to colorless

### Example 5

| Chemical Component | Weight in grams |
|---|---|
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3,5-dimethoxyphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3,5-dimethoxyphenyl)-1-(3H)-isobenzofuranone, sodium hydroxide, deionized water, sodium chloride and liquid germall plus were added and mixture was further stirred for 4 hours at room temperature.

Color of the solution: Teal

Color change: Teal to colorless

### Example 6

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3,5-dimethylphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3,5-dimethylphenyl)-1-(3H)-isobenzofuranone, sodium hydroxide, deionized water, sodium chloride and liquid germall plus were added and mixture was further stirred for 4 hours at room temperature.

Color of the solution: Purple

Color change: Purple to colorless

### Example 7

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3,6-diimethylphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3,6-diimethylphenyl)-1-(3H)-isobenzofuranone, sodium hydroxide, deionized water, sodium chloride and liquid germall plus were added and mixture was further stirred for 4 hours at room temperature.

Color of the solution: Blue

Color change: Blue to colorless

### Example 8

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3-ethylphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3-ethylphenyl)-1-(3H)-isobenzofuranone, sodium hydroxide, deionized water, sodium chloride and liquid germall plus were added and mixture was further stirred for 4 hours at room temperature.

Color of the solution: Magenta

Color change: Magenta to colorless

### Example 9

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-hydroxy-3-isopropylphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-hydroxy-3-isopropylphenyl)-1-(3H)-isobenzofuranone, sodium hydroxide, deionized water, sodium chloride and liquid germall plus were added and mixture was further stirred for 4 hours at room temperature.

Color of the solution: Pink

Color change: Pink to colorless

### Example 10

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3-methoxyphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3-methoxyphenyl)-1-(3H)-isobenzofuranone, sodium hydroxide, deionized water, sodium chloride and liquid germall plus were added and mixture was further stirred for 4 hours at room temperature.

Color of the solution: Blue

Color change: Blue to colorless

### Example 11

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-2,3,5-trimethylphenyl)- | 0.5 |
| 1-(3H)-isobenzofuranone | |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-2,3,5-trimethylphenyl)-1-(3H)-isobenzofuranone, sodium hydroxide, deionized water, sodium chloride and liquid germall plus were added and mixture was further stirred for 4 hours at room temperature.

Color of the solution: Teal

Color change: Teal to colorless

### Example 12

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| Bromo-thymolphthalein | 0.5 |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. Bromo-thymolphthalein, sodium hydroxide, deionized water, sodium chloride and liquid germall plus was added and mixture was further stirred for 4 hours at room temperature.

Color of the solution: Teal

Color change: Teal to colorless

### Example 13

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| Bromo-o-cresolphthalein | 0.5 |
| Sodium hydroxide | 1 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. Bromo-o-cresolphthalein, sodium hydroxide, deionized water, sodium chloride and liquid germall plus was added and mixture was further stirred for 4 hours at room temperature.

Color of the solution: Purple

Color change: Purple to colorless

### Example 14

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3-sec-butylphenyl)- | 0.5 |
| 1-(3H)-isobenzofuranone | |
| Sodium hydroxide | 1.00 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3-sec-butylphenyl)-1-(3H)-isobenzofuranone, sodium hydroxide, deionized water, sodium chloride and liquid germall plus were added and mixture was further stirred for 4 hours at room temperature.

Color of the solution: Pink

Color change: Pink to colorless

### Example 15

| | Weight |
|---|---|
| Chemical Component | in grams |
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| 3,3-bis-(4-Hydroxy-3-nitrophenyl)- | 0.5 |
| 1-(3H)-isobenzofuranone | |
| Sodium hydroxide | 1.00 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. 3,3-bis-(4-Hydroxy-3-nitrophenyl)-1-(3H)-isobenzofuranone, sodium hydroxide, deionized water, sodium chloride and liquid germall plus were added and mixture was further stirred for 4 hours at room temperature.

Color of the solution: Yellow

Color change: Yellow to colorless

### Example 16

| Chemical Component | Weight in grams |
|---|---|
| Stepan-Mild LSB | 28.00 |
| Steol CS-330 | 21.00 |
| Amphosol HCA | 12.00 |
| Ninol 40-CO | 1.5 |
| Dow Corning 190 | 0.5 |
| Sodium chloride | 0.41 |
| m-Nitrophenol | 0.5 |
| Sodium hydroxide | 1.00 |
| Deionized water | 34.99 |
| Liquid Germall Plus | 0.1 |

Stepan-Mild LSB, Steol CS-330, Amphosol HCA, Ninol 40-CO and Dow Corning 190 were stirred at room temperature for 45 minutes till it became homogeneous. m-Nitrophenol, sodium hydroxide, deionized water, sodium chloride and liquid germall plus were added and mixture was further stirred for 4 hours at room temperature.

Color of the solution: Golden yellow

Color change: Golden yellow to colorless

### Example 17

| Chemical Component | Weight in grams |
|---|---|
| Commercial Shampoo^{a,b,c} | 50 |
| Dye^{d} | 0.5 |
| Sodium hydroxide | 2 equivalent^{e} |
| Commercial Shampoo^{a}: Commercial shampoos used include but not limited to Suave (Unilever), Clairol Herbal (Proctor and gamble), and Aussie (Redmond). | |
| Commercial Shampoo^{b}: Suave ingredients include but not limited to water, ammonium laurly sulfate, ammonium laureth sulfate, ammonium chloride, cocamid MEA, fragrance (Parrum), PEG-5 cocamid, hyroxypropyl methyl cellulose, tetrasodium EDTA, DMDM hydantoin, citric acid, panthenol (provitamin B5), propylene glycol, methylchloroisothiazolinone, methylisothiazolinone, passionflower (Paciflora edulis) extract, | |
| # peppermint (Mentha piperita) extract, rose (Rose canina) extract, lavender (Lavadula angustifolia) extract, D & C Violet No. 2. | |
| Commercial Shampoo^{c}: Clairol Herbal ingredients include but not limited to water, stearyl alcohol, cyclopentasiloxane, cetyl alcohol, stearmidopropyl dimethylamine, hydrolyzed wheat protein, starch, Rubus fruticocus (blackberry) fruit extract, Persea gratissima (Avocado) fruit extract, Magnifera indica (Mango) fruit extract, fragrance, dimethicone, glutamic acid, benzyl alcohol, EDTA, | |
| # methylchloroisothiazolinone, methylisothiazolinone, Red 33, orange 4, Violet 2 | |
| Commercial Shampoo^{c}: Aussie ingredients include but not limited to water, ammonium lauryl sulfate, ammonium laureth sulfate, glycol distearate, Aloe barbadensis leaf, Anigoxanthos flavidus flower extract, tocopherol acetate, panthenol, cocamid MEA, dimethicone, fragrance, cetyl alcohol, sodium chloride, sodium citrate, sodium benzoate, guar hydroxypropyltriammoniumchloride, disodium EDTA, hydrogenated | |
| # polydecene, citric acid, trimethylolpropane tricaprylate/tricaprate, methylchloroisothiazolinone, methylisothiazolinone, ammonium xylenesulfonate, Yellow 5, Red 4, kangaroo paw flower extract, vitamin E acetate | |

Dyed^{d}: All the above dyes were used.
Equivalent^{e}: 2 Equivalent of sodium hydroxide required for generating initial color of the formulation but if pH drops to neutral or acidic, more sodium hydroxide required to generate/keep color for short period, depending on the dye and pH of the formulation.

In a beaker containing commercial shampoo was added dye and sodium hydroxide. The reaction mixture was further stirred for 2 hours at room temperature.

Test Procedure:

Colored shampoo from any of the above examples was taken in both the hands and rubbed for 30 seconds to 1 minute, the respective color disappears.

Soap Bar Examples:

### Example 1

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| Thymolphthalein | 0.5 |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, thymolphthalein, deionized water and Liquid Germall Plus and further stirred for 10 minutes. The batch was allowed to stand for 1 hour without stirring at 76° C. The product transferred to soap molds which solidified.

Color of the soap bar: Blue

Color change: Blue to colorless

### Example 2

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| o-Cresolphthalein | 0.5 |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, o-cresolphthalein, deionized water and Liquid Germall Plus and further stirred for 10 minutes. The batch was allowed to stand for 1 hour without stirring at 76° C. The product transferred to soap molds which solidified.

Color of the soap bar: Magenta

Color change: Magenta to colorless

### Example 3

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3-phenylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3-phenylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Purple

Color change: Purple to colorless

### Example 4

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3,5-diisopropylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3,5-diisopropylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Violet

Color change: Violet to colorless

### Example 5

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3,5-dimethoxyphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3,5-dimethoxyphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Teal

Color change: Teal to colorless

### Example 6

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3,5-dimethylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3,5-dimethylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Purple

Color change: Purple to colorless

### Example 7

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3,6-diimethylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3,6-diimethylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Blue

Color change: Blue to colorless

### Example 8

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3-ethylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3-ethylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Magenta

Color change: Magenta to colorless

### Example 9

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-hydroxy-3-isopropylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-hydroxy-3-isopropylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Pink

Color change: Pink to colorless

### Example 10

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3-methoxyphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3-methoxyphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Blue

Color change: Blue to colorless

### Example 11

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-2,3,5-trimethylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-2,3,5-trimethylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Teal

Color change: Teal to colorless

### Example 12

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| Bromo-thymolphthalein | 0.5 |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, bromo-thymolphthalein, deionized water and Liquid Germall Plus and further stirred for 10 minutes. The batch was allowed to stand for 1 hour without stirring at 76° C. The product transferred to soap molds which solidified.

Color of the soap bar: Teal

Color change: Teal to colorless

### Example 13

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| Bromo-o-cresolphthalein | 0.5 |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, bromo-o-cresolphthalein, deionized water and Liquid Germall Plus and further stirred for 10 minutes. The batch was allowed to stand for 1 hour without stirring at 76° C. The product transferred to soap molds which solidified.

Color of the soap bar: Purple

Color change: Purple to colorless

### Example 14

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3-sec-butylphenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3-sec-butylphenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Pink

Color change: Pink to colorless

### Example 15

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| 3,3-bis-(4-Hydroxy-3-nitrophenyl)- | 0.5 |
| 1-(3H)-isobenzofuranone | |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, 3,3-bis-(4-Hydroxy-3-nitrophenyl)-1-(3H)-isobenzofuranone, deionized water and Liquid Germall Plus and further stirred for 10 minutes. The batch was allowed to stand for 1 hour without stirring at 76° C. The product transferred to soap molds which solidified.

Color of the soap bar: Yellow

Color change: Yellow to colorless

### Example 16

| Chemical Component | Weight in grams |
|---|---|
| Amphosol HCG | 5.0 |
| Alpha-Step MC-48 | 5.0 |
| Steol CS-330 | 12.0 |
| Stearic acid | 15.00 |
| Propylene glycol | 15.00 |
| Glycerin | 5.00 |
| Sodium chloride | 1.5 |
| Triethanolamine | 1.5 |
| m-Nitrophenol | 0.5 |
| Sodium hydroxide | 6.0 |
| Deionized water | 33.4 |
| Liquid Germall Plus | 0.1 |

A mixture of propylene glycol, glycerin, Amphosol HCG, Steol CS-330, were stirred and heated at 60° C. till it became homogeneous. Stearic acid was slowly added to stirring mixture at 60° C. The temperature of the reaction mixture was raised to 76° C. Sodium hydroxide was slowly added under stirring at 76° C. The temperature of the mixture was further raised to 85° C. and stirred until the mixture became homogeneous (30 minutes). Alpha-Step MC-48 was added at 85° C. under stirring, followed by addition of triethanolamine, sodium chloride, m-nitrophenol, deionized water and Liquid Germall Plus and further stirred for 10 minutes. The batch was allowed to stand for 1 hour without stirring at 76° C. The product transferred to soap molds which solidified.

Color of the soap bar: Golden yellow

Color change: Golden yellow to colorless

### Example 17

| Chemical Component | Weight in grams |
|---|---|
| Commercial Bar Soap^{a} | 50 |
| Dye^{b} | 0.5 |
| Sodium hydroxide | 2 equivalent |

Commercial Bar Soap^{a}: Commercial bar soap used include but not limited to Dove (Unilever)
Dye^{b}: All the above dyes were used.

In a beaker containing crushed commercial soap bar was added dye and sodium hydroxide. The reaction mixture was further stirred for 2 hours at room temperature.

Test Procedure:

Colored soap bar from any of the above examples was taken in the hands and rubbed with the addition of water for 30 seconds to 1 minute, the respective color disappears.

Although the present invention has been described with reference to preferred embodiments, persons skilled in the art will recognize that changes may be made in form and detail without departing from the spirit and scope of the invention.

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A bodywash comprising:
a surfactant; and
an acid-base indicator comprising: 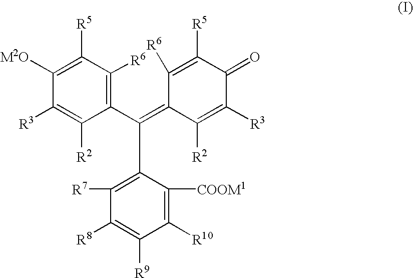
wherein
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom; and
M¹ and M² are each independently a hydrogen atom, a metal ion or an ammonium ion, provided that at least one of M¹ or M² is a metal ion or an ammonium ion.

2. The bodywash of claim 1, wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

3. The bodywash of claim 1, wherein R², R³, R⁴, R⁵, R⁶, R⁷, R⁸, R⁹ and R¹⁰ are hydrogen atoms, or R² is hydrogen, R³ is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³ is Br, R⁵ is an isopropyl and R⁶, R⁷, R⁸, R⁹ and R¹⁰ are hydrogen atoms.

4. The bodywash of claim 1, wherein R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms or R², R³ and R⁵ methoxide and R⁵, R⁶, R⁷, R⁸, R⁹ and R¹⁰ are all hydrogen atoms, or R², R³ an R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

5. The bodywash of claim 1, further comprising a base.

6. The bodywash of claim 5, wherein the base is a metal hydoxide.

7. The bodywash of claim 1, wherein the bodywash is in the form of a shampoo.

8. The bodywash of claim 1, where the bodywash is in the form of a bar of soap.

9. The bodywash of claim 1, wherein the bodywash is in the form of a liquid soap.

10. The composition of claim 1, wherein the bodywash is in the form of a gel.

11. A method to cleanse a portion of an individual's body comprising application of a colored composition comprising a surfactant and
an acid-base indicator comprising: 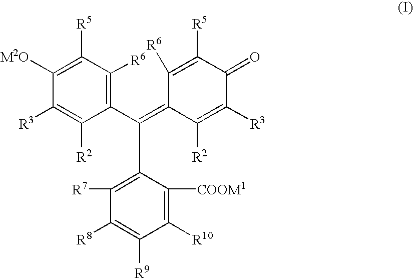
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom; and
M¹ and M² are each independently a hydrogen atom, a metal ion or an ammonium ion, provided that at least one of M¹ or M² is a metal ion or an ammonium ion, to the individual's body; and
treatment of the body portion with the composition for a sufficient period of time, such that a color change of the composition occurs.

12. The method of claim 11, wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

13. The method of claim 11, wherein R², R³, R⁵, R⁶, R⁷, R⁸, R⁹ and R¹⁰ are hydrogen atoms, or R² is hydrogen, R³ is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³ is Br, R⁵ is an isopropyl and R⁶, R⁷, R⁸, R⁹ and R¹⁰ are hydrogen atoms.

14. The method of claim 11, wherein R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

15. The method of claim 11, wherein the color change is from color to clear.

16. The method of claim 15, wherein the period of time is between about 10 seconds and about 2 minutes.

17. The method of claim 11, wherein the body portion is a hand.

18. The method of claim 11, wherein the body portion is the scalp.

19. A packaged personal care cleansing system comprising a composition and instructions, wherein the composition comprises:
a surfactant; and
an acid-base indicator comprising: 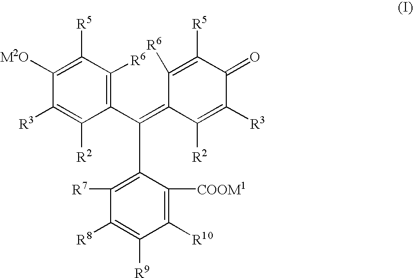
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom; and
M¹ and M² are each independently a hydrogen atom, a metal ion or an ammonium ion, provided that at least one of M¹ or M² is a metal ion or an ammonium ion; and
the instructions are for use of the system, wherein the individual washes with the composition for a sufficient period of time whereby a color change indicates that the cleansing period is complete.

20. The system of claim 19, wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

21. The system of claim 19, wherein R², R³, R⁵, R⁶, R⁷, R⁸, R⁹ and R¹⁰ are hydrogen atoms, or R² is hydrogen, R³ is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³ is Br, R⁵ is an isopropyl and R⁶, R⁷, R⁸, R⁹ and R¹⁰ are hydrogen atoms.

22. The system of claim 19, wherein R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

23. The system of claim 19, wherein the color change is from a color to clear.

24. The system of claim 19, wherein the composition is applied to the individual's hand.

25. The system of claim 19, wherein the composition is applied to the individual's scalp.

26. A method to remove pathogens from an individual's skin, comprising the step of:
applying a colored composition to the individual's body, the composition comprising a surfactant; and
an acid-base indicator comprising: 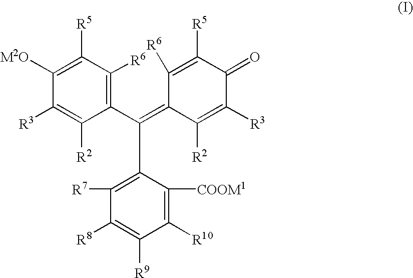
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom; and
M¹ and M² are each independently a hydrogen atom, a metal ion or an ammonium ion, provided that at least one of M¹ or M² is a metal ion or an ammonium ion.

27. The method of claim 26, wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

28. The method of claim 26, wherein R², R³, R⁴, R⁵, R⁶, R⁷, R⁸, R⁹ and R¹⁰ are hydrogen atoms, or R² is hydrogen, R³ is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³ is Br, R⁵ is an isopropyl and R⁶, R⁷, R⁸, R⁹ and R¹⁰ are hydrogen atoms.

29. The method of claim 26, wherein R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

30. The method of claim 26, further comprising the step of rubbing the composition applied to the body surface for a sufficient period of time, whereby the colored composition has a loss of color.

31. The method of claim 30, further comprising the step of rinsing the portion of the body treated with the colored composition with water.

32. A waterless hand cleaner comprising:
an emulsifiable lipophilic organic solvent compatible with skin;
a quantity of water present from 5 to 70 total weight percent;
a surfactant that forms a gelatinous emulsion between the organic solvent and the quantity of water; and
an acid-base indicator comprising: 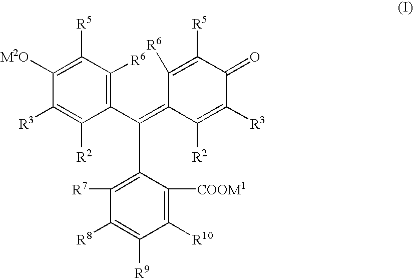
wherein
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom; and
M¹ and M² are each independently a hydrogen atom, a metal ion or an ammonium ion, provided that at least one of M¹ or M² is a metal ion or an ammonium ion.

33. The hand cleaner of claim 32, wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

34. The hand cleaner of claim 32, wherein R², R³, R⁵, R⁶, R⁷, R⁸, R⁹ and R¹⁰ are hydrogen atoms, or R² is hydrogen, R³ is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³ is Br, R⁵ is an isopropyl and R⁶, R⁷, R⁸, R⁹ and R¹⁰ are hydrogen atoms.

35. The hand cleaner of claim 32, wherein R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

36. The hand cleaner of claim 32, further comprising a base.

37. The hand cleaner of claim 36, wherein the base is a metal hydroxide.
